(19) United States
(12) Patent Application Publication
(10) Pub. No.: US 2026/0215905 A1
(43) Pub. Date: Jul. 30, 2026

(54) METHODS AND APPARATUSES FOR ATTACHING SOFT TISSUE TO BONE WITH KNOTLESS REPAIR

(71) Applicant: Biomet Sports Medicine, LLC, Warsaw, IN (US)

(72) Inventors: Ryan A. Kaiser, Leesburg, IN (US); Christopher M. Palese, Warsaw, IN (US); Nyree Masoian, San Ramon, CA (US); Stephanie Zahler, Warsaw, IN (US); Preston McCurdy, Fort Wayne, IN (US)

(21) Appl. No.: 19/450,546

(22) Filed: Jan. 15, 2026

Related U.S. Application Data
(60) Provisional application No. 63/749,469, filed on Jan. 24, 2025.

(51) Int. Cl. A61F 2/08

(52) U.S. Cl. CPC A61F 2/0811; A61F 2002/0852; A61F 2220/0075

(57) ABSTRACT

A method and apparatus for attaching soft tissue to bone using a knotless repair system. The assembly includes a flexible tubular anchor with a passage, a first suture strand with an eyelet, and a second suture strand forming an adjustable loop. The first suture strand shuttles a free portion of the second suture strand or a third suture strand through the loop, which adjusts to capture the free portion, securing the free portion to the anchor. The loop can be internal or external to the anchor. The method involves passing a repair suture strand through the loop and adjusting the loop size prior to, during or after inserting the anchor into the bone.

### CLAIM OF PRIORITY

This application claims the benefit of U.S. Provisional Patent Application Serial No. 63/749,469, filed on January 24, 2025, the benefit of priority of which is claimed hereby, and which is incorporated by reference herein in its entirety.

### FIELD OF THE DISCLOSURE

The present disclosure relates to methods and apparatuses used for a tissue repair surgery including apparatuses for attaching soft tissue to bone.

### BACKGROUND OF THE DISCLOSURE

In the human body, tissue can require repair. Such tissue includes bone, muscles, tendons, ligaments and cartilage. Forceful twisting, trauma or rotation of the knee, shoulder (or other joint) can tear or otherwise damage tissue. A surgical repair of the tissue may be required. Such repair can include suturing tissue and anchoring to bone. Various assemblies have been developed for facilitating suturing and are effective for their intended purposes. Nevertheless, improvements to tissue repair devices for facilitating suturing are still desirable.

### SUMMARY

Various tissue repair devices including sutures and anchors have been developed. Although these tissue repair devices are generally effective, there is a constant need for improvement particularly regarding facilitating a reduction in the number or knots used for such repairs. Additionally, some tissue repairs are complex and require the deployment of multiple anchors to anchor sutures and repair tissue in multiple locations. The present application simplifies complex procedures by enabling various configurations of repair strand routing and securing, while maintaining strong fixation.

The present inventors have recognized that surgical complexity and procedure time can be reduced with the present tissue repair devices, which include an adjustable loop(s) that can be coupled with prior to, during or after insertion of the suture anchor into bone. Thus, the present tissue repair devices can have the suture anchor remain in situ such as within a bone hole or adjacent the bone hole as the suture(s) are coupled therewith. Alternatively or additionally, the present tissue repair devices can have repair suture(s) coupled thereto prior to being placed in situ. This improves the versatility and utility of the present tissue repair devices.

According to one example, the adjustable loop can extend from the suture anchor and includes a cinch strand (also called a zip strand herein) configured to adjust the size of the loop as desired. This configuration using the adjustable loop improves versatility and enhances the number and type of sutures that can be coupled to the suture anchor.

According to one example, the present application includes an assembly and methods for securing soft tissue to bone during surgical procedures. The assembly can include a flexible tubular anchor, a first suture strand (e.g., a shuttle strand) that creates an eyelet and a second suture strand (e.g., a repair strand, a cinching strand, etc.) forms an adjustable loop using a saddle section. The loop can be configured to capture either (or both) of a free portion of the second suture strand or a third suture strand. According to one example, the assembly can have the loop is captured within a passage of the flexible tubular anchor. This configuration allows the first suture strand to shuttle a free portion of the second suture strand through the loop and cinch the free portion of the second suture strand within the flexible tubular anchor. According to yet another example, the loop can extend from the flexible tubular anchor, with the second suture strand including a cinching portion that extends through at least a portion of the flexible tubular anchor and is configured to adjust the loop size.

The present inventors have further recognized that patient recovery can be enhanced with the use of repair suture strands that have a bulked section that includes a biological material. More particularly, the present inventors have developed bulked and non-bulked sections in the suture strands, where the bulked section features a relatively larger cross-sectional area as the result of combining two or more suture strands together. This bulked section can be designed to integrate a material such as biological material(s) that are not as structurally supportive into the repair strand in combination with more structurally supportive material(s). Thus, for example, the present application contemplates biological material such as but not limited to ActivBraid™ Collagen Co-Braid by Zimmer Biomet of Warsaw, Indiana, used in combination with biocompatible materials that are more structurally robust in nature. Such biocompatible material can include but are not limited to a synthetic material(s). The biological material(s) can either captured within or forming an outer braid of the bulked section.

The present inventors contemplate methods of securing tissue to bone that can allow surgeons to pass repair suture strands through the adjustable loop and adjust the loop size before, during, or after inserting the flexible tubular anchor into bone. The present inventor contemplate use in various soft tissue repairs including in rotator cuff repairs, where multiple flexible tubular anchors can be strategically placed in medial and lateral rows, with repair strands passed back and forth through adjustable loops to create desired tissue fixation patterns.

### BRIEF DESCRIPTION OF THE DRAWINGS

FIG. 1 illustrates a tissue repair assembly including a suture anchor coupled to a plurality of suture strands according to one example of the present application.

FIG. 2 is an enlarged view of a portion of a repair suture strand that is one of the plurality of suture strands of FIG. 1.

FIG. 3 is an enlarged view of portions of the tissue repair assembly including suture anchor and portions of the plurality of suture strands of FIG. 1.

FIG. 4 illustrates the tissue repair assembly of FIG. 3 with the suture anchor removed to show features of the plurality of suture strands including an adjustable loop that would otherwise be positioned within the suture anchor according to one example of the present application.

FIG. 5 illustrates the tissue repair assembly of FIGS. 1-4 with the suture anchor shown in a highly schematic manner and in the assembly in the process of having a repair suture strand that forms a loop about tissue being passed through the adjustable loop via a shuttle suture strand.

FIG. 5A is an enlarged view of portions of the tissue repair assembly of FIG. 5.

FIG. 6 further illustrates the tissue repair assembly of FIGS. 1-5A with the suture anchor shown in a highly schematic manner with a loop reduced to be fully cinching the repair suture strand.

FIG. 7 illustrates another example of a tissue repair assembly including a suture anchor coupled to a plurality of suture strands including a repair suture strand that has a bulked section and a non-bulked section according to one example of the present application.

FIG. 8 is a cross-sectional view of an example of the repair suture strand of FIG. 7 at a transition between the bulked section and the non-bulked section illustrating two different material suture strands of two different materials used in the bulked section according to one example of the present application.

FIG. 9 is a plan view of the repair suture strand of FIG. 8 at the transition between the bulked section and the non-bulked section with a tighter braid configuration according to one example of the present application.

FIG. 10 is a plan view of the repair suture strand of FIG. 8 at the transition between the bulked section and the non-bulked section with a looser braid configuration according to one example of the present application.

FIG. 11 illustrates yet another example of a tissue repair assembly including a suture anchor coupled to a plurality of suture strands with the plurality of strands being tape strands and having at least a repair suture strand with a bulked section according to one example of the present application.

FIG. 12 illustrates a tissue repair assembly including a suture anchor coupled to a plurality of suture strands including a cinching suture strand that forms an adjustable loop extending from and positioned substantially entirely outside the suture anchor according to one example of the present application.

FIGS. 13A and 13B show a tissue repair assembly including a suture anchor coupled to a plurality of suture strands including a cinching suture strand that forms two or more adjustable loops extending from and positioned substantially entirely outside the suture anchor according to one example of the present application.

FIGS. 14-16 illustrate a rotator cuff repair using two of the tissue repair assemblies of FIG. 12 positioned in a medial row and two of the tissue repair assemblies of FIG. 1 placed in a lateral row according to one example of the present application.

FIGS. 17-19 illustrates a rotator cuff repair using two of the repair assemblies of FIGS. 1 positioned in a medial row and two of the tissue repair assemblies of FIG. 12 placed in a lateral row according to one example of the present application.

FIGS. 20 and 21 illustrate a rotator cuff repair using two of the tissue repair assemblies of FIG. 12 positioned in a medial row and two other hard suture anchors in the lateral row according to one example of the present application.

FIGS. 22-24 illustrate a rotator cuff repair using at least two tissue repair assemblies that each include a suture anchor that is double loaded with the suture constructs discussed herein including the adjustable loop of FIG. 12 and the internal cinching loop within the anchor discussed in FIGS. 1-6.

FIG. 25 shows a flow diagram of a method of securing tissue to bone according to one example of the present application.

FIG. 26 shows a flow chart of a method of securing tissue to bone according to one example of the present application.

FIG. 27 shows a tissue repair assembly including a suture anchor coupled to a plurality of suture strands that combines the concepts of FIGS. 1 and 12 according to one example of the present application.

FIG. 28 illustrates a tendon repair using at least one tissue repair assembly that includes a suture anchor that is loaded with a plurality of suture strands as discussed herein including the adjustable loop of FIG. 12 where the tissue is captured by a cinched adjustable loop according to one example of the present application.

FIG. 29 illustrates a tendon repair using at least one tissue repair assembly that includes a suture anchor that is loaded with suture constructs discussed herein including with a plurality of suture strands as discussed herein including the multiple adjustable loops of FIGS. 13A, 13B or 27, wherein the tissue is captured by two cinched loops according to one example of the present application.

FIG. 30 shows a flow chart of a method of securing tissue to bone according to one example of the present application.

Corresponding reference characters indicate corresponding parts throughout the several views. Inserters in the drawings are not necessarily drawn to scale. The configurations shown in the drawings are merely examples, and should not be construed as limiting the scope of the invention in any manner.

### DETAILED DESCRIPTION

To repair tissue (including both soft tissue and bone) in the human body a surgeon can deploy suture anchors into the tissue. Suture anchors and suture strands can be used to reattach muscle to bone, for example. Although the following description is related generally to suture anchors, sutures and suture constructs using deformable anchors in connection with an exemplary rotator cuff repair techniques, it will be understood that the devices and methods discussed herein can also be applicable to other appropriate surgical procedures, such as, for example, a shoulder labral repair, a labral tear in a hip joint, a Pectoralis Major Repair, Achilles Tendon Repair, Quadriceps Tendon Repair, Patellar Tendon Repair, Triceps Tendon Repair, Lateral Collateral Ligament Repair/Reconstruction, Medial Collateral Ligament Repair/Reconstruction, a Remplissage, a Lateral Ankle Repair, etc. Therefore, it will be understood that the following exemplary discussions are not intended to limit the scope of the present teachings or claims herein.

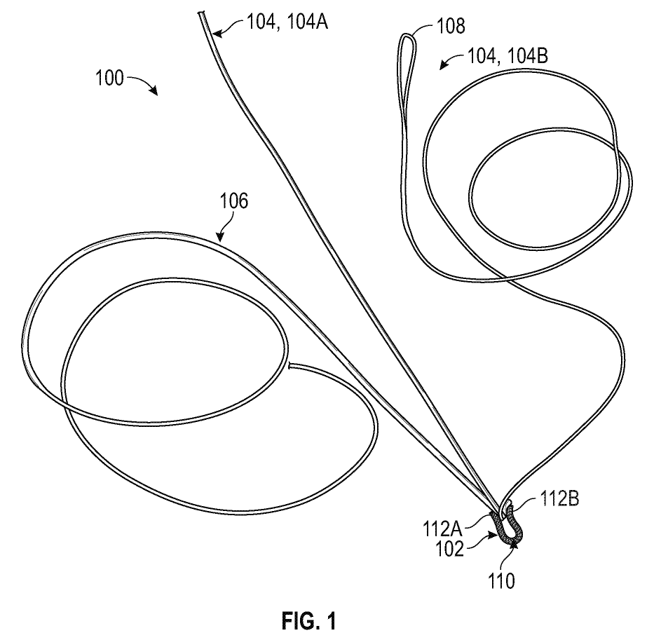

FIG. 1 illustrates a tissue repair assembly 100. The tissue repair assembly 100 can include an anchor 102, a first suture strand 104 and a second suture strand 106. The first suture strand 104 can include a first portion 104A and a second portion 104B with an eyelet 108. The anchor 102 can include a saddle section 110 and wings 112A and 112B.

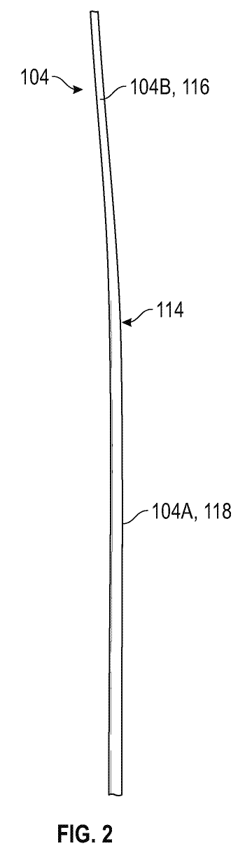

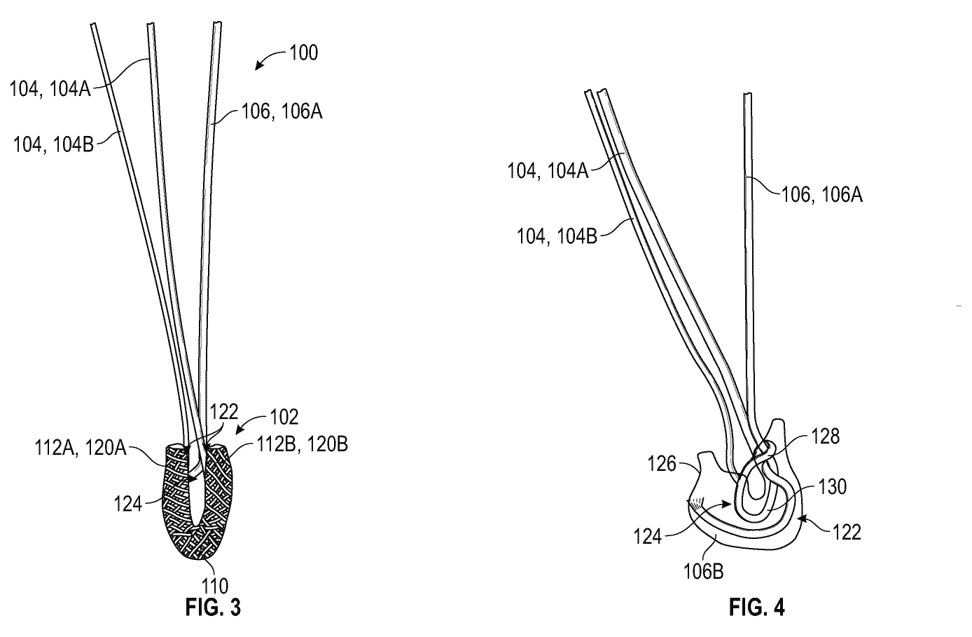

The anchor 102 is shown in a bone delivery position as would be engaged by a delivery device (not shown). In this position, the anchor 102 is U-shaped with the saddle section 110 positioned between the wing 112A and the wing 112B. The anchor 102 can be a soft anchor as further discussed in reference to FIG. 3. The first suture strand 104 and the second suture strand 106 can have any desired construction and/or material composition as known in the art. Thus, the first suture strand 104 and/or the second suture strand 106 can be monofilament or multi-filament, can be non-braided, can be circular in cross-section or tape (having major flat portion) in cross-section, can be made of resorbable or non-resorbable materials, can be perforated, woven/braided from biocompatible or biocomposite materials or fibers, such as, for example, polymer, nitinol, polyester, polyethylene, cotton, silk, ActivBraid™ or other suitable biological, natural or synthetic materials. As shown in FIG. 2, according to one example, the first suture strand 104 can include a transition 114 between the first portion 104A and the second portion 104B. This transition 114 can also be between a first style of suture strand 116 (e.g., round braid) and a second style of suture strand 118 (e.g., tape, different colored braid, different material, etc.).

As shown in FIG. 1, the first suture strand 104 can be configured as a shuttle suture strand to pass through at least a portion of a passage formed by the anchor 102. The second suture strand 106 can be coupled to the anchor 102 and can configured to be received by the eyelet 108 that is part of the second portion 104B. The second suture strand 106 can be a repair suture strand for coupling with tissue (e.g., by wrapping, being stitched through, etc.). The second suture strand 106 can pass through at least a portion including up to an entirety of the passage of the anchor 102. The second suture strand 106 can include a saddle (discussed subsequently) forming a loop. This loop can extend through the anchor 102. The eyelet 108 can be configured to capture another free suture such as the second suture strand 106 or a third suture (not shown). Indeed, the eyelet 108 can be configured to capture multiple suture strands (e.g., second suture strand 106, a third suture strand and/or a third suture strand and a fourth suture strand, a third, fourth and fifth suture strands, etc.) as desired. Although described as being configured to couple with suture strands, the eyelet 108 can be configured to couple with other components if desired.

In operation, the first suture strand 104 can be configured to shuttle a free portion of the second suture strand 106 or the third suture strand through the loop of the second suture strand 106. The loop formed by the second suture strand 106 and passing through the anchor 102 is adjustable to change a size thereof and is configured to capture the free portion of the second suture strand 106 or the third suture strand to couple the free portion of the second suture strand 106 or the third suture strand to the anchor 102.

Turning to FIG. 3, FIG. 3 illustrates portions of the tissue repair assembly 100 including the anchor 102 with the saddle section 110 and the wings 112A and 112B, the first suture strand 104 including the first portion 104A and the second portion 104B and the second suture strand 106.

In the example of FIG. 3, the anchor 102 can be a soft anchor having a flexible tubular construction according. The anchor 102 can be configured as a sleeve with the wings 112A and 112B forming a first end 120A and a second end 120B, respectively. The anchor 102 can form a passage 122 that extends internally from adjacent to or at the wings 112A and 112B. The flexible nature of the anchor 102 allows the anchor 102 to be deformable. Examples of the present anchor 102 construct are described, for example, in Application Serial No. 15/482,106 (now U.S. Patent No. 10,499,902), Application Serial No. 15/654,386 (now U.S. Patent No. 10,695,045), Application Serial No. 16/251,342 (now U.S. Patent No. 11,116,495), Application Serial No. 14/055,172 (now U.S. Patent No. 9,724,090) and Application Serial No. 18/133,848, the entire disclosures of which are incorporated herein by reference. The anchor 102 can be configured as an elongate member with the passage 122 internal thereto extending between the wings 112A and 112B. The anchor 102 can have a suture construct (e.g., can be formed of larger sized braided suture or other suitable material(s)), for example. Thus, the anchor 102 can be made of resorbable or non-resorbable materials, including braided suture, sponges and sponge-like materials in solid form, perforated materials, woven/braided from biocompatible or biocomposite materials or fibers, such as, for example, polymer, polyester, polyethylene, cotton, silk, or other natural or synthetic materials.

The anchor 102 can have properties that allow the anchor to be flexible/deformable to change shape (e.g., curve, bend, collapse, etc.). In this regard, the anchor 102 can be, for example, compliant, flexible, foldable, squashable, squeezable, deformable, limp, flaccid, elastic, low-modulus, soft, spongy or perforated, or have any other characteristic property that allows it to change shape. For simplicity, the anchor 102 is simply described herein as flexible, deformable or soft in capturing such properties. In some aspects, the anchor 102 can be coated with biological or biocompatible coatings, can include other biological material incorporated therein in a bulked section (as further described and shown), and/or can be soaked in platelets and other biologics, which can be easily absorbed by the anchor 102. In one exemplary configuration, the anchor 102 can be formed from a strand of No. 5 braided polyester suture. The anchor 102 can be made of resorbable or non-resorbable materials as discussed above. In other words, multiple fibers can be braided together to form a hollow braided suture having a longitudinal passage (discussed subsequently).

The anchor 102 can also be referred to herein as a soft suture anchor(s). The anchor 102 is referred to as “soft” herein as they are formed of material(s) that are flexible and/or deformable, such as a suture sleeve or other suture material. These soft suture anchors can be constructed as tubes that are collapsible once tension is applied to the suture. Use of soft suture anchors made of deformable material can be preferable to using a hard-plastic anchor, made from a relatively hard material, such as polyether ether ketone (PEEK). For example, in cases where the suture pulls through the tissue, the suture anchor can be dislodged within the joint or surgical space. In such instances, chances of damage is reduced with the soft suture anchor as compared with the hard suture anchor. Soft anchors are known and include the JuggerKnot® Soft Anchor and JuggerKnotless® Soft Anchor distributed or sold by Zimmer Biomet of Warsaw, IN. However, the methods discussed subsequently discuss used of flexible tubular anchors in combination with hard-plastic anchors.

The first suture strand 104 at the second portion 104B and the second suture strand 106 that is part of an adjustable closed loop 124 can be passed through a first opening in a wall of the anchor 102 at or adjacent the wing 112A into the passage 122. The first suture strand 104 at the first portion 104A and the second suture strand 106 at the free portion 106A can be passed through a second opening in the wall of the anchor 102 and along the passage 122 at or adjacent the wing 112B. The loop 124 passes through the passage 122 and emerges from the anchor 102 to pass between the first opening and the second opening. Thus, the loop 124 includes at least a portion thereof that is exposed from the anchor 102.

The first suture strand 104 and the second suture strand 106 can be guided along the passage 122 formed by the anchor 102. Although shown adjacent the ends 120A and 120B in FIG. 3, the first and/or second openings can be positioned anywhere intermediately between the wings 112A and 112B of the anchor 102. For example, the first and second openings can at a distance of, for example, one-quarter length from ends. It will be appreciated that the openings can be apertures or voids in the woven fabric of the anchor 102, such that the openings do not disrupt or break the weave of the anchor 102 when made of braided or woven material. Further, portions of the anchor 102 such as the saddle section 110 can be between the first and second ends (the wings 112A and 112B). Saddle section 110 can have the U-shape for engagement with a delivery device for insertion into bone as discussed previously. The wings 112A and 112B and indeed the saddle section 110 can provide resistance for securing the anchor 102 relative to the bone.

The first suture strand 104 can be the shuttle suture strand. The first suture strand 104 can form a suture construct having free ends that formed by the first portion 104A and the second portion 104B. The eyelet 108 (FIG. 1) can be part of the second portion 104B such as at an end thereof. Once captured by the eyelet 108 (FIG. 1), the second suture strand 106, the third suture (not shown), etc. can be shuttled by the first suture strand 104 by pulling on the first portion 104A. This can pass the second portion 104B of the first suture strand 104 and captured suture(s) such as the free portion 106A through the anchor 102. Once the free portion 106A emerges from the second opening at or adjacent the wing 112B (at or adjacent the second end 120B) from pulling the first portion 104A, both ends of the second suture strand 106 can be grasped and the loop 124 can be adjusted (reduced) to cinch. Thus, the loop 124 is adjustable to change a size thereof and is configured to capture the free portion 106A of the second suture strand 106 and/or the third suture strand (not shown in example of FIG. 3) to couple the free portion 106A of the second suture strand 106 ad/or the third suture strand to the anchor 102. More specifically, as shown in FIG. 3, the loop 124 is captured by the anchor 102 within the passage 122 but has a portion thereof that emerges externally from the anchor 102 passing between the first opening and the second opening (between the wing 112A and the wing 112B in the example of FIG. 3). The first suture strand 104 can be configured to shuttle the free portion 106A of the second suture strand 106 through the loop 124. The loop 124 is adjustable to close about itself and thereby cinch and capture the free portion 106A of the second suture strand 106 within the anchor 102.

FIG. 4 shows the loop 124 in further detail as the anchor 102 (FIG. 3) has been removed with only the outline of a wall 126 thereof shown for illustrative purposes. FIG. 4 additionally shows first suture strand 104 including the first portion 104A and the second portion 104B and the second suture strand 106 with the free portion 106A as discussed previously. FIG. 4 additionally illustrates the second suture strand 106 includes a dead strand 106B that is an end of the second suture strand 106 that is positioned within the anchor 102 (FIG. 3). The loop 124 formed by the second suture strand 106 includes a wrapped section 128 and a saddle section 130.

FIG. 4 shows the loop 124 is almost entirely internal to the passage 122 within the wall 126. The loop 124 is formed by the wrapped section 128 which passes the dead strand 106B around and through the interior of the loop 124. The saddle section 130 of the loop 124 follows the shape of the anchor 102 (FIG. 3). The first suture strand 104 passes through the loop 124.

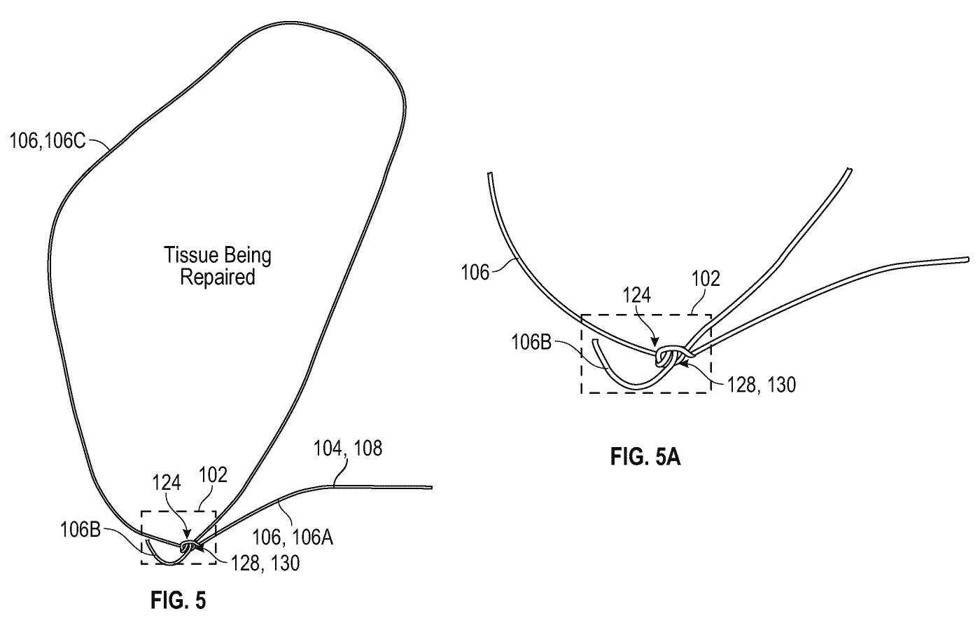

FIG. 5 and 5A illustrate the tissue repair assembly 100 with the anchor 102 shown in a highly schematic manner to illustrate operation of the loop 124 during capture and coupling. FIG. 5 shows the dead strand 106B and a second loop 106C formed about the tissue being captured. In FIG. 5, the free portion 106A of the second suture strand has been wrapped around tissue and is then coupled to the first suture strand 104 (e.g., via the eyelet 108). The first suture strand 104 has been passed through the anchor 102 and the loop 124 carrying the free portion 106A along with it through the anchor 102 and the loop 124. The loop 124 has been reduced in size by tensioning the second suture strand 106. This tensioning and reduction of the loop 124 reduces the saddle section 130 to be positioned adjacent with the wrapped section 128. Thus, FIGS. 5 and 5A show loop 124 is adjustable to close about itself and thereby cinch and capture the free portion 106A of the second suture strand 106 within the anchor 102.

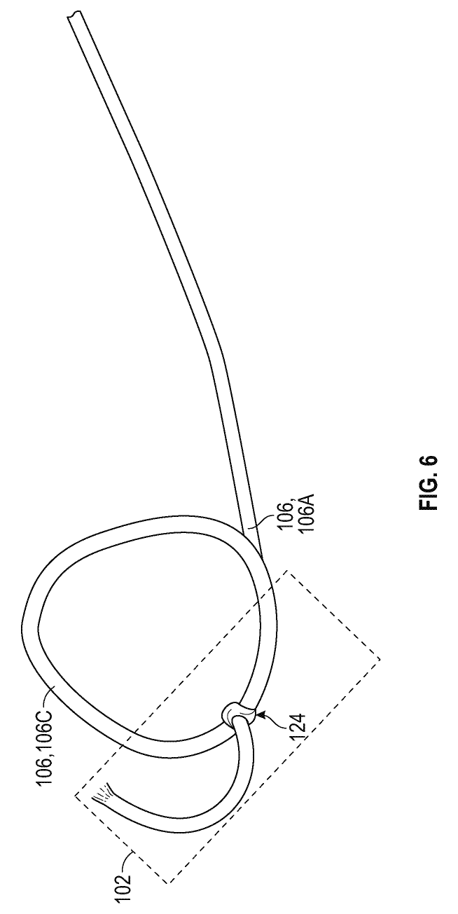

FIG. 6 illustrates the second loop 106C further reduced as well as the loop 124 fully cinched to couple the free portion 106A of the second suture strand 106 to the anchor 102. It should be noted that although FIGS. 5-6 show the anchor 102 as a rectangle the closing of the loop 124 as well as the construction of the anchor 102 would result in deformation of the anchor 102 to change shape resulting in fixation of the anchor 102 with bone. Examples of this deformation are discussed and illustrated in Application Serial No. 15/482,106 (now U.S. Patent No. 10,499,902), Application Serial No. 15/654,386 (now U.S. Patent No. 10,695,045), Application Serial No. 16/251,342 (now U.S. Patent No. 11,116,495), Application Serial No. 14/055,172 (now U.S. Patent No. 9,724,090) and Application Serial No. 18/133,848 and further patent application filings assigned to the applicant.

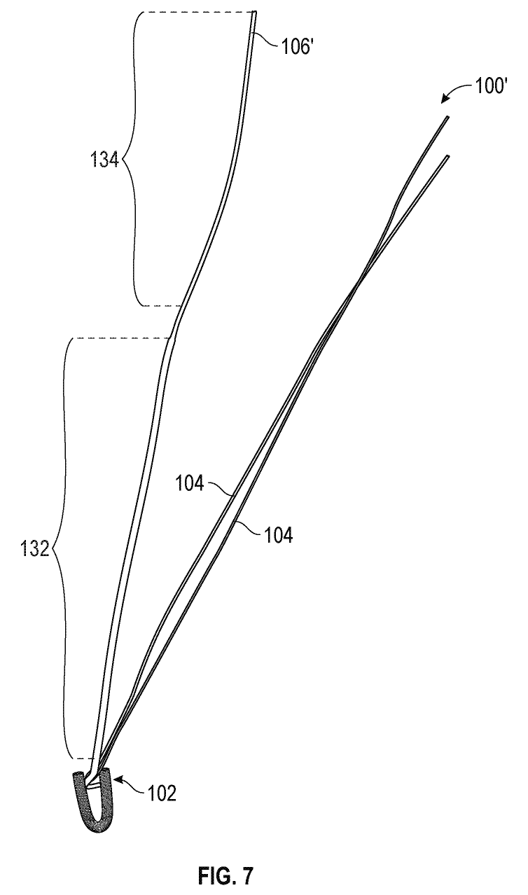

FIG. 7 illustrates a tissue repair assembly 100’ of similar construction that of the tissue repair assembly 100 of FIGS. 1-6. Thus, the tissue repair assembly 100’ can include the anchor 102, the first suture strand 104 and a second suture strand 106’. The second suture strand 106’, which can be the repair strand as discussed previously, can differ in that it can include a bulked section 132 and a non-bulked section 134. The bulked section 132 can be positioned more closely adjacent the anchor 102 than the non-bulked section 134. The bulked section 132 can have a relatively larger cross-sectional area than the non-bulked section 134. Although the present application provides examples of the bulked section and non-bulked section in the context of the tissue repair assemblies as examples, it should be recognized this concept (bulked and non-bulked suture sections) can be applied to any suture and need not be applied to specifically to tissue repair assemblies.

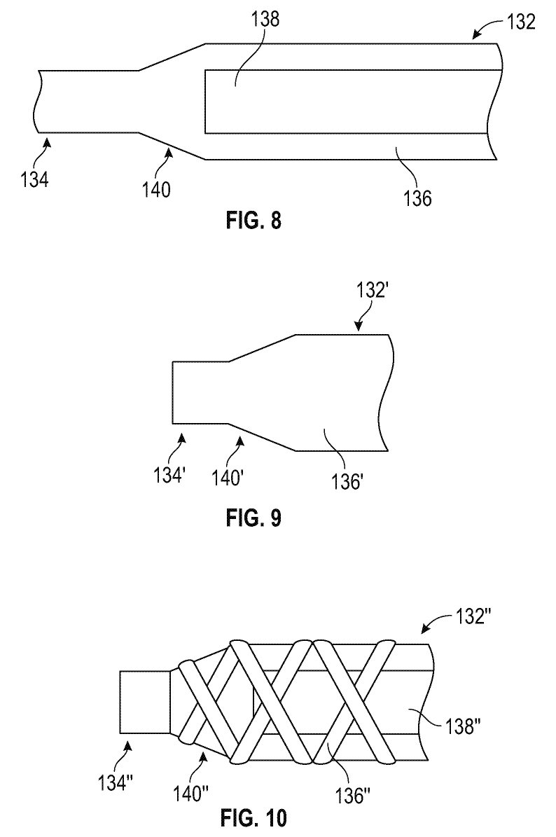

FIGS. 8-12B provide different examples of the bulked section. As shown in cross-section of FIG. 8, the bulked section 132 can be created by stuffing or otherwise inserting a second suture strand 138 inside a first suture strand 136. FIG. 8 illustrates a transition 140 such as a tapered section between the bulked section 132 and the non-bulked section 134 illustrating the first suture strand 136 and the second suture strand 138. The first suture strand 136 and the second suture strand 138 can be constructed of different material(s) from one another. Thus, at least two different materials are used in the bulked section 132 in the example of FIG. 8. For example, the first suture strand 136 can form an exterior of the bulked section 132 and can be biocompatible material(s) that are more structurally robust in nature than the second suture strand 138. Such biocompatible material can include but are not limited to a synthetic material(s) such as polymers (e.g., polyester, polyethene, etc.), nitinol and other material. The second suture strand 138 can form a core of the bulked section 132 and can be a biological non-braided material (e.g., can be resorbable, can be naturally occurring) or can be other suitable material(s)). Alternatively, the second suture strand 138 forming the core can be the biocompatible material that is more structurally robust than the first suture strand 136 and the first suture strand 136 that forms an outer part of the bulked section 132 can be formed of the biological material such as described for example in US Patent Nos. 11,020,509 and 11,338,057, the entire contents of each of which are incorporated herein by reference. Thus, FIG. 8 shows the bulked section 132 includes the biological material in combination with the synthetic material according to one example. Additionally, the biological material can be captured within or can form an outer braid (outer portion including surfaces) of the bulked section 132.

FIG. 9 shows another example of the bulked section 132’, the transition 140’ and the non-bulked section 134’. A first suture strand 136’ can form an exterior of the bulked section 132’. The bulked section 132’ is again formed by biological material in combination with the synthetic material, for example. However, in FIG. 9, the biological material (not shown in FIG. 9) is entirely encapsulated by the synthetic material such that the synthetic material forms the outer surface of the bulked section 132’. This is a referred to herein as a tight braid configuration.

FIG. 10 shows yet another example of the bulked section 132’’, the transition 140’’ and the non-bulked section 134’’. A first suture strand 136’’ can form an exterior of the bulked section 132’’. The bulked section 132’’ is again formed by biological material in combination with the synthetic material. In FIG. 10, the biological material of the second suture strand 138’’ is exposed through openings in the synthetic material (the first suture strand 136’’). Thus, even though the synthetic material forms the outer surface of the bulked section 132’’ the biological material of the core (the second suture strand 138’’) is still exposed. This is a referred to herein as a loose braid configuration.

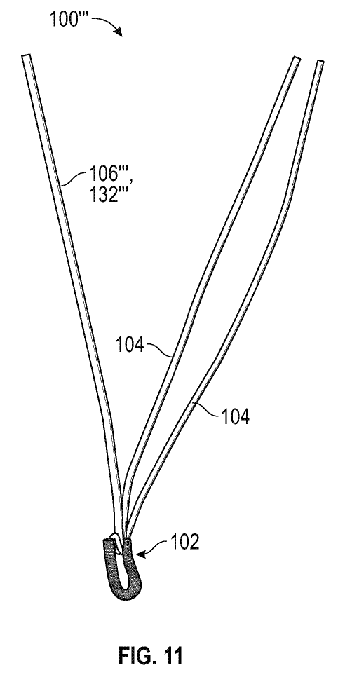

FIG. 11 illustrates a tissue repair assembly 100’’’ of similar construction that of the tissue repair assembly 100 of FIGS. 1-6 and tissue repair assembly 100’ of FIG. 7. Thus, the tissue repair assembly 100’’’ can include the anchor 102, the first suture strand 104 and a second suture strand 106’’’. The second suture strand 106’’’, which can be the repair strand as discussed previously, can differ in that it can include the bulked section 132’’’ formed of overlying and/or weaving tape strands. FIG. 11 does not show a non-bulked section but one can be utilized if desired. The bulked section 132’’’ can be positioned more closely adjacent the anchor 102 and can extend therefrom.

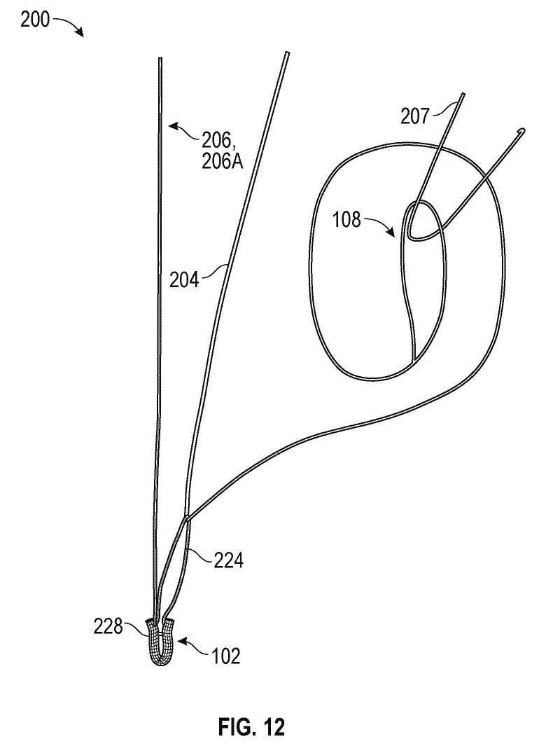

FIG. 12 shows a tissue repair assembly 200 that differs from those discussed previously, although aspects and components of the tissue repair assembly 200 can be shared with the assemblies discussed previously. In FIG. 12, the tissue repair assembly 200 can include the anchor 102, a first suture strand 204 including an eyelet 108 and a second suture strand 206. Optionally, but not required in some embodiments, the tissue repair assembly 200 can include a third suture strand 207.

The first suture strand 204 can be an optional shuttle strand, for example. Unlike previously discussed shuttle strand, the first suture strand 204 need not pass through the anchor 102. The second suture strand 206, rather than being a repair strand, can be a cinching or zip strand for capturing and cinching the third suture strand 207 or tissue to the anchor 102. Such cinching of the third suture strand 207 or tissue can be either above or below a cortical layer of bone. The second suture strand 206 can include an adjustable loop 224 and a second portion 206A that acts to change the size of the loop 224. The adjustable loop 224 extends from the anchor 102 and can be substantially entirely or entirely positioned outside of the anchor 102 (and in some cases can be positioned outside the joint space) having a wrapped section 228 adjacent a first opening of the anchor 102. The second portion 206A can extend through the anchor 102 interior along the passage. According to some examples, the first suture strand 104 can be used to shuttle the third suture strand 207 through the loop 224. However, according to other examples the third suture strand 207 can be passed through the loop 224 without the aid of the first suture strand 104. The third suture 207 can be passed through the shuttle strand 204 before or after inserting the anchor 102 into bone.

According to one method of use, the first suture strand 204 can be passed through the loop 224 and the third suture strand 207 can be coupled to the first suture strand 204 via the eyelet 108. The surgeon can pull the second portion 206A to cinch/adjust the loop 224 down against the anchor 102. The third suture strand (repair suture(s)) can be coupled to the anchor with cinching of the loop 224. Thus, the example of FIG. 12 illustrates that the first suture strand 204 is configured to shuttle the third suture strand 207 through the loop 224. The loop 224 is adjustable to change a size thereof and is configured to capture the third suture strand 207 to couple the third suture strand 207 to the anchor 102.

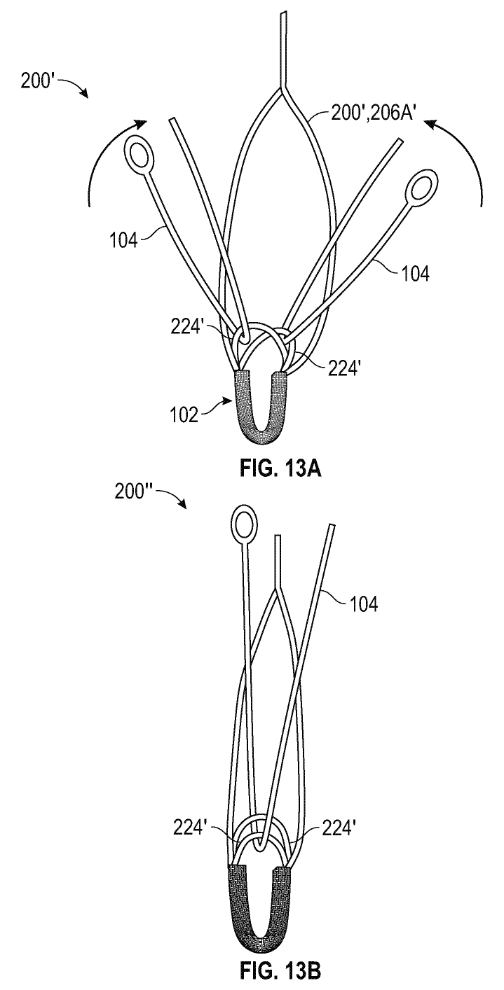

FIG. 13A shows a tissue repair assembly 200’ of similar to that of the tissue repair assembly 200 of FIG. 12 but differing in that the second suture strand 204 can be passed through the anchor 102 multiple times to form two or more loops 224’. The second suture strand 206’, rather than being a repair strand, can be a cinching or zip strand for capturing and cinching a third suture strand (not shown) to the anchor 102. The second suture strand 206’ can include the two or more adjustable loops 224’ and a second portion 206A’ that acts to change the size of the two or more adjustable loops 224’. The adjustable loops 224’ each extend from the anchor 102 and can be substantially entirely or entirely positioned outside of the anchor 102. The second portion 206A’ can extend through the anchor 102 interior along the passage. According to some examples, the first suture strand 104 (two first sutures are shown in FIG. 13A) can be used to shuttle a third suture strand (not shown) and/or a fourth suture strand (not shown) through each of the two or more loops 224’. However, according to other examples, at least the third suture strand 207 can be passed through one or more of the two or more loops 224 without the aid of the first suture strand 104. Additionally or alternatively, tissue such as a tendon can be passed through the loop 224’.

FIG. 13B shows a tissue repair assembly 200’’ similar to that of the tissue repair assembly of FIG. 13A but differing in that only a single first suture strand 104 or single third suture is passed through two (or all) of the two or more loops 224’.

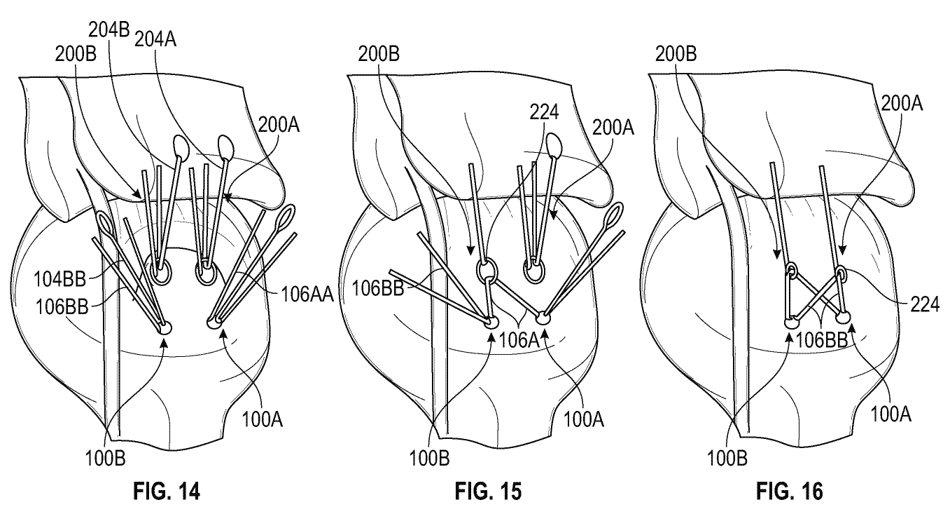

FIGS. 14-16 illustrate a rotator cuff repair using two of the tissue repair assemblies 200A and 200B of FIG. 12 positioned in a medial row and two of the tissue repair assemblies 100A and 100B of FIG. 1 placed in a lateral row. As shown in FIG. 15, the surgeon can pass a second suture strand 106AA (a repair strand also shown in FIG. 14) of a first of the tissue repair assembly 100A, through a first suture strand 204B (shown in FIG. 14) (a shuttle strand) and adjustable loop 224 of a diagonally arranged tissue repair assembly 200B. As further show in FIG. 15, the surgeon then passes the same second suture strand 106AA (the repair strand) of the first of the tissue repair assembly 100A through an opposite first suture strand 104BB (again shown in FIG. 14) of the second tissue repair assembly 100B. In FIG. 15, after passage through the anchor, the second suture strand 106AA is now locked in (coupled to) the anchor of the tissue repair assembly 100B opposite the tissue repair assembly 200B. As shown in FIG. 16, the surgeon can then repeats the process with a remaining second repair strand 106BB (also shown initially in FIGS. 14 and 15) of the second of the tissue repair assembly 100B. This second repair strand 106BB can be passed diagonally to and through adjustable loop 224 of the tissue repair assembly 200A such as by using the first suture strand 204A (shown in FIG. 14). The second suture strand 106BB is then locked in (coupled to) the anchor of the tissue repair assembly 100A opposite the tissue repair assembly 200A.

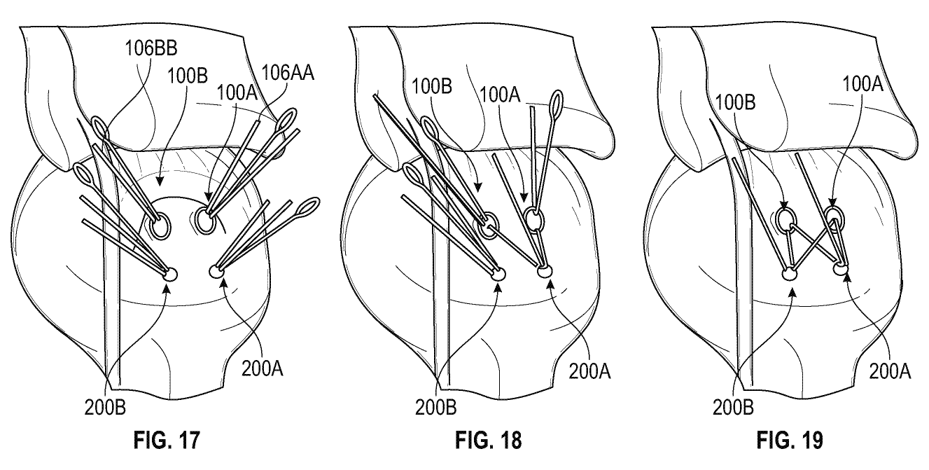

FIGS. 17-19 illustrates a rotator cuff repair using two of the repair assemblies 100A and 100B of FIGS. 1 positioned in a medial row and two of the tissue repair assemblies 200A and 200B of FIG. 12 placed in a lateral row. However, variations of this pattern are possible include use of a single anchor for one of the medial row or the lateral row or use of three or more anchors for one or both of the medial row and the lateral row. The process of diagonally passing repair strands (the second suture strands 106AA and 106BB) described above is repeated as is returning to the original row to lock the second suture strands 106AA and 106BB (FIG. 17) down.

Thus, FIGS. 14-19 disclose methods of performing a rotator cuff repair. These methods can include: inserting a first flexible tubular anchor and second flexible tubular anchor in a medial row, inserting a third flexible tubular anchor and a fourth flexible tubular anchor in a lateral row, passing a first repair strand from the third flexible tubular anchor through an adjustable loop coupled to and extending from the first flexible tubular anchor or passing a second repair strand from the first flexible tubular anchor through an adjustable loop coupled to and extending from the third flexible tubular anchor and securing the first repair strand to the fourth flexible tubular anchor or securing the second repair strand to the second flexible tubular anchor.

According to further examples, the methods of FIGS. 14-19, can further include: passing a third flexible tubular anchor from the fourth flexible tubular anchor through an adjustable loop coupled to and extending from the second flexible tubular anchor or passing a fourth repair strand from the second flexible tubular anchor through an adjustable loop coupled to and extending from fourth flexible tubular anchor and securing the third repair strand to the third flexible tubular anchor or securing the fourth repair strand to the first flexible tubular anchor. The methods of can further include adjusting a size of the loop coupled to and extending from the first flexible tubular anchor and the loop coupled to and extending from the third flexible tubular anchor before, during, or after the inserting the first flexible tubular anchor into the medial row and the third flexible tubular anchor into the lateral row.

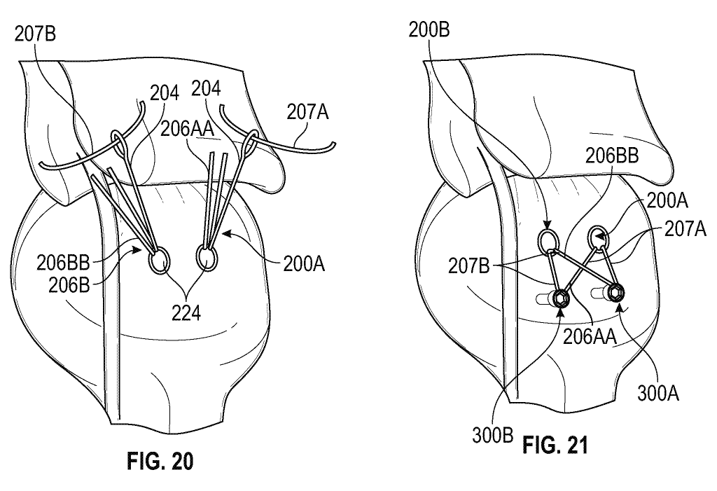

FIGS. 20 and 21 illustrate a rotator cuff repair using two of the tissue repair assemblies 200A and 200B of FIG. 12 positioned in a medial row and two hard suture anchors 300A and 300B (shown FIG. 21 only) in the lateral row. The surgeon can pass third suture strands 207A and 207B through the first suture strand 204 (shown in FIG. 20 only) and the adjustable loops 224 (shown in FIG. 20 only) of the tissue repair assemblies 200A and 200B. The surgeon can cinch the adjustable loops 224 to capture the third suture strands 207A and 207B. The surgeon can then pass the third suture strands 207A and 207B and/or the second suture strands 206AA and 206BB in a diagonal manner to the opposite hard suture anchors 300A and 300B as shown in FIG. 21.

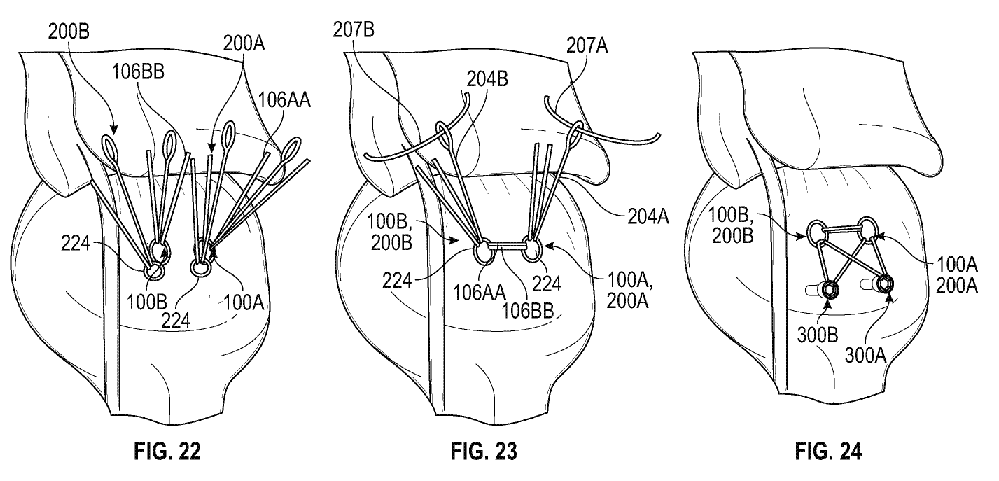

FIGS. 22-24 illustrate yet another rotator cuff repair using at least two tissue repair assemblies 100A, 100B, 200A and 200B that each include a suture anchor that is double loaded with the suture constructs discussed herein including the adjustable loop 224 of FIG. 12 and the internal cinching loop within the anchor discussed in FIGS. 1-6 (not shown). FIGS. 22-24 illustrate use of these double loaded anchor constructs and suture strands for tissue repair. The method can include passing repair strands 106AA and 106BB (FIGS. 22 and 23) between and through the anchors via shuttles (not numbered) as shown in FIG. 23. The surgeon can then use the first repair strands 204A and 204B (FIG. 23) as shuttles to pass a third suture strand 207A and 207B (FIGS. 23) through the adjustable loops 224. The remaining free sutures can be coupled to hard suture anchors 300A and 300B as shown in FIG. 24.

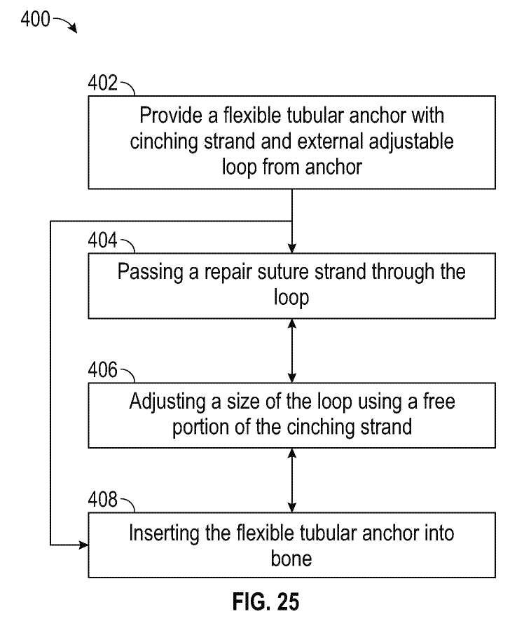

FIG. 25 is a flow diagram of a method 400 of securing tissue to bone. The method 400 can include providing 402 a flexible tubular anchor having cinching strand coupled thereto. The cinching strand forms a loop extending from and positioned substantially entirely externally to the flexible tubular anchor. The method 400 can include passing a repair suture strand through the loop with a shuttle strand. The method at step 404 can include adjusting a size of the loop using a free portion of the cinching strand. The method at step 406 can include inserting the flexible tubular anchor into the bone. Various steps of the method 400 including the passing the repair suture strand through the loop with the shuttle strand and the adjusting the size of the loop can be performed before, during, or after the inserting the flexible tubular anchor into the bone.

According to further examples, the method 400 can include securing the repair suture strand to a second anchor and inserting the second anchor into the bone. The method 400 can optionally further include: providing a second flexible tubular anchor having the repair suture strand coupled thereto; inserting the second flexible tubular anchor into the bone; passing the repair suture strand through the loop; and securing the repair suture strand to the second flexible tubular anchor. The securing the repair suture strand to the second flexible tubular anchor optionally includes closing a second loop about itself to cinch and capture the repair suture strand within the second flexible tubular anchor. Alternatively, the securing the repair suture strand to the second flexible tubular anchor optionally includes: providing a second cinching strand coupled to the second flexible tubular anchor, the second cinching strand forms a second loop extending from the second flexible tubular anchor; passing the repair suture strand through the second loop with a shuttle strand; adjusting the size of the second loop using a free portion of the second cinching strand; and inserting the second flexible tubular anchor into the bone. Optionally, at least one of the repair suture strand includes a bulked section and a non-bulked section. The bulked section includes a relatively larger cross-sectional area than the non-bulked section. The bulked section includes a biological material in combination with a synthetic material. The method 400 can optionally further include cinching and capturing the repair suture strand with a third loop captured by one of the flexible tubular anchor or the second flexible tubular anchor.

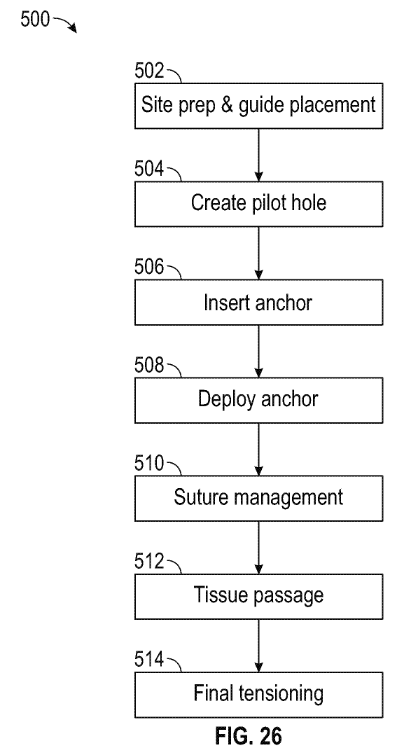

FIG. 26 illustrates an exemplary repair using one of the tissue repair assemblies of FIGS. 1-12 for a shoulder labral repair or rotator cuff repair (discussed subsequently in the following paragraph). This surgical method 500 can include at 502 Site Preparation and Guide Placement. This can include immobilizing the labrum and create a bone surface; passing a guide through cannula to a place on glenoid rim. Optionally, site preparation of the bone surface can include the bone surface be roughened to create a bleeding bone surface while maintaining cortical bone integrity at anchor site(s). The surgeon passes a guide through the cannula and positions it on the glenoid rim, with the option to use a centering sleeve through the curved guide for enhanced drilling stability. The method 500 can include at 504 Pilot Hole Creation. This can include inserting a drill bit into power drill; inserting a drill into drill guide and advancing drill until contact with guide handle. The method 500 can include at 506 Anchor Insertion. This step can include maintaining the guide position over pilot hole; removing the centering sleeve if used; inserting a soft anchor (part of one of the tissue repair assemblies of FIGS. 1-12) through guide into pilot hole; inserting until flush with a back of the guide; and verifying a proper depth by checking mark alignment. The method 500 at 508 can include Anchor Deployment. This step can include pulling back on anchor inserter handle to begin setting anchor; removing a suture cap to release sutures; removing an anchor inserter and guide; pulling on both sutures together to set anchor; and verifying suture color or pattern. The method 500 at 510 can include Suture Management. This step can include using a suture grasper to transfer a suture strand to a posterior portal and separating suture strands using instrument tip. The method 500 at 512 can include creating a tissue passage. This step can include inserting a suture passer through anteroinferior cannula; passing through labral tissue inferior to anchor position; advancing a wire into joint; and retrieving a loop through posterior portal. The method 500 can include at step 514 final tensioning. This can include loading repair suture strand(s) through an eyelet of a first suture strand outside posterior portal; pulling the first suture strand to shuttle repair suture strand through labral tissue; pulling the first suture strand to shuttle the repair suture strand; and pulling the repair strand for final tensioning.

According to another option, the method 500 can include pulling back on the anchor inserter handle to begin setting the anchor. The suture is released from the handle by removing the suture cap, followed by removal of the anchor inserter and guide. A light pull on all sutures together sets the anchor, with specific attention to verify that the white/blue suture slides while noting that the blue/black repair strand will not slide. The method 500 can include a suture grasper transfers the blue/black repair strand to the anterosuperior portal. Using a suture passer or similar device inserted through the anteroinferior cannula, the surgeon passes through the labral tissue at the desired repair location. The nitinol wire is advanced manually into the joint once the suture passer tip penetrates the tissue, and the wire loop is retrieved through the anterosuperior portal. The method can include use of the suture anchors and suture techniques discussed herein to facilitate tensioning, here approximately 2 inches of the blue/black suture limb is folded through the white suture loop outside the anteroinferior portal. The white/blue shuttle strand is pulled to shuttle the blue/black suture around the tissue and through the anchor body's locking mechanism. Tension is applied to the blue/black strand until the repair is complete, and finally, the suture tail is cut flush with a flush suture cutter.

Further examples contemplate other surgical methodology and techniques. Such surgical technique can include preparation of the bone surface, which may be roughened to create a bleeding bone surface while maintaining cortical bone integrity at anchor sites. The guide is placed securely and perpendicular to the bone at the desired location. A pilot hole is then created by inserting the drill bit into the power drill to the proximal laser-etch line and advancing it through the drill guide until contact is made. After removing the drill while maintaining guide position, the suture anchor such as one of those disclosed herein is inserted through the guide and into the pilot hole, with careful malleting of the inserter handle until the laser etch line is flush with the back of the inserter handle to avoid weakening the bone. The anchor is then deployed by pulling back on the anchor inserter handle and removing the white suture cap to release the suture. The inserter handle is pulled directly out from the guide, followed by removal of the drill guide and gentle pulling of all sutures together to properly set the anchor. For suture management, up to two free sutures can be folded through the white shuttle loop, ensuring approximately 2 inches of each free suture is folded over. The white/blue tape side of the shuttle strand is pulled to pass the free suture into the anchor body loop. The black suture limb is then pulled to cinch the loop and secure the free sutures into the bone. The suture limbs are individually loaded through the side load passer and passed through the tissue. For double-row repairs, arthroscopic knots are tied from posterior to anterior, with the suture strands spanning over the lateral aspect of the tendon and secured with a knotless anchor (examples provided above). The repair is completed according to the surgeon's preferred technique, with any remaining or excess suture being cut.

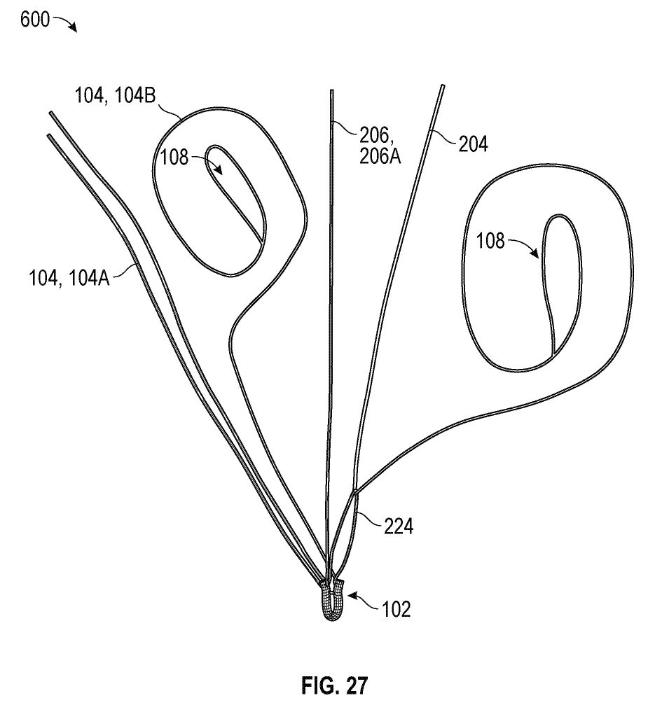

FIG. 27 illustrates constructs discussed such as in FIG. 12 and FIG. 1 can be combined into a single anchor 102 having both an external adjustable loop 224 and an internally adjustable loop (not shown as captured by the single anchor 102 as previously shown). The example of FIG. 27 thus illustrates a tissue repair assembly 600. The tissue repair assembly 600 can include the anchor 102, a first suture strand 104, a second suture strand 106, a third suture strand 204 (described as the first suture strand in reference to FIG. 12) including an eyelet 108 and a fourth suture strand 206 (described as the second suture strand in reference to FIG. 12). The first suture strand 104 can include a first portion 104A and a second portion 104B with an eyelet 108. The fourth suture strand 206 can include the adjustable loop 224 and the second portion 206A that acts to change the size of the loop 224 as previously described.

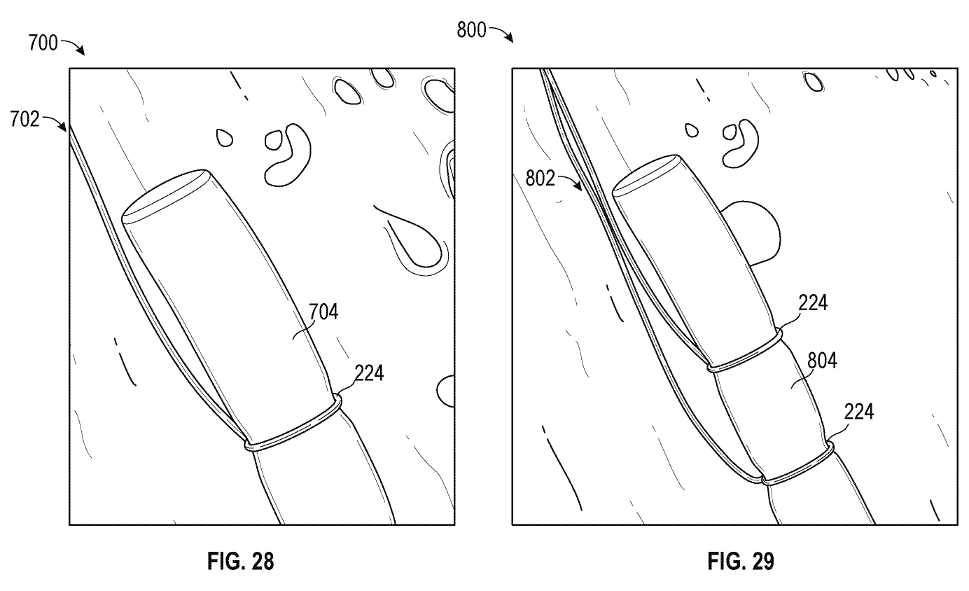

FIG. 28 shows a tendon repair 700 using at least one tissue repair assembly 702 that includes a suture anchor (not shown) that is loaded with a plurality of suture strands as discussed herein including suture strands and the adjustable loop 224 of FIG. 12 where the tendon 704 is captured by the adjustable loop 224 when cinched.

FIG. 29 shows a tendon repair 800 using the tissue repair assembly 802 that includes a suture anchor (not shown) that is loaded with suture constructs discussed herein including with a plurality of suture strands as discussed and the multiple adjustable loops (e.g., the loop 224 of FIG. 27) and the additional internal loop that was not shown in FIG. 27 but previously shown and described in FIGS. 1-6, wherein the tissue 804 is captured by two loops, including the loop 224, that are both cinched.

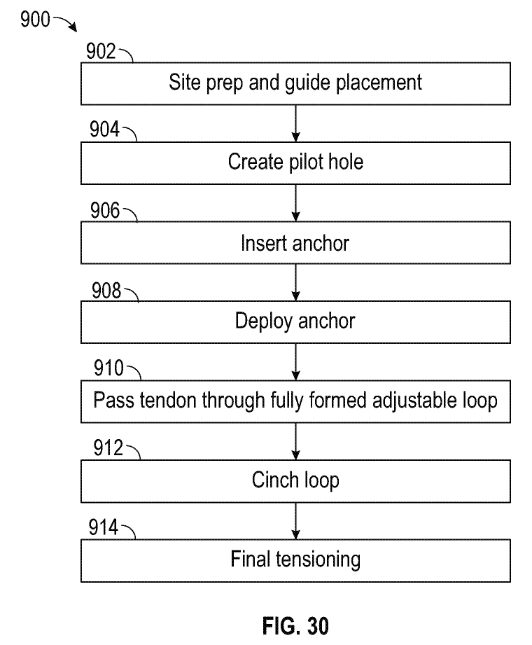

FIG. 30 illustrates a method 900 for repair of tissue such as the tendon repair of FIGS. 28 or 29 for the securing tissue to bone. The method 900 can include providing a flexible tubular anchor having cinching strand coupled thereto. The cinching strand forms a loop extending from and positioned substantially entirely externally to the flexible tubular anchor. The method 900 can include passing the tissue through the loop with or without a shuttle strand. The method can include adjusting a size of the loop using a free portion of the cinching strand. The method at step 906 can include inserting the flexible tubular anchor into the bone. Various steps of the method 900 including the passing the tissue through the loop with the shuttle strand and the adjusting the size of the loop can be performed before, during, or after the inserting the flexible tubular anchor into the bone.

The surgical method 900 can include at 902 Site Preparation and Guide Placement similar as to previously described in method step 502 of FIG. 26. The method 900 can include at 904 Pilot Hole Creation similar to step 504 described in FIG. 26. This can include inserting a drill bit into power drill; inserting a drill into drill guide and advancing drill until contact with guide handle. The method can include at 906 Anchor Insertion similar to as previously described at step 506 of FIG. 26. The method 900 at 908 can include Anchor Deployment as previously described at step 508 of FIG. 26. The method 900 at 910 can passing tissue through a collapsable adjustable loop of the through the loop with or without a shuttle strand. The method 900 at 912 can include cinching the loop to capture the tissue therein (see examples of FIGS. 28 and 29). The method 900 can include at step 914 final tensioning.

The present disclosure relates to a soft tissue to bone fixation system comprising anchors designed for orthopedic surgical procedures. The loop concepts discussed herein are an all-suture anchor system that is configured to provide secure attachment of soft tissue to bone across multiple anatomical locations and surgical applications. The system finds particular utility in rotator cuff repair procedures, though the anchors are indicated for use in a variety of shoulder, knee, foot, ankle, and elbow procedures. Shoulder applications include biceps tenodesis, shoulder instability repair, and rotator cuff repair. Knee applications include medial patellofemoral ligament (MPFL) repair, patellar tendon repair, medial collateral ligament (MCL) repair, and quadriceps tendon repair. Foot and ankle applications include Achilles tendon repair, medial and lateral repair and reconstruction, plantar plate repair, mid-foot and forefoot repair, and metatarsal ligament or tendon repair or reconstruction. Elbow applications include lateral epicondylitis repair and biceps tendon reattachment. The breadth of these indications demonstrates the versatility of the anchor construct and the surgical technique described herein.

The systems discussed herein can be all-suture anchors that are available with OsseoCoat™ technology, which distinguishes certain embodiments from standard soft anchor configurations. The soft anchors with OsseoCoat™ technology are structurally identical to the standard soft anchors except for the presence of bioceramics embedded in the anchor portion of the device. The hydroxyapatite and bioceramic particles provide a surface coating comprised of materials with osteoconductive properties. These osteoconductive properties have been documented in scientific literature, including histomorphometric studies of hydroxyapatite coated and uncoated porous titanium bone implants, studies of surface characteristics and biological responses to hydroxyapatite coatings applied by various methods, and comprehensive reviews of bioactive glass materials and their interactions with bone tissue. The OsseoCoat™ technology is intended to promote integration of the anchor with the surrounding bone tissue over time.

The anchor construct itself can comprise multiple suture elements that work in concert to facilitate the passage and fixation of repair materials within a bone pilot hole. A first suture element can be a shuttle pull strand, which is colored white and blue in a bicolor configuration. This shuttle pull strand can be connected to a shuttle loop, which is colored white. The shuttle loop can form an opening through which additional repair sutures or tapes may be passed after the anchor has been deployed within the bone. A second suture element can be a cinching strand, which is colored blue and black in a bicolor configuration. The cinching strand can be connected to an adjustable loop at the anchor body. When tension is applied to the cinching strand, the adjustable loop reduces in diameter, thereby capturing and securing any repair materials that have been passed through the shuttle loop and into the adjustable loop. The color coding of the various suture elements can facilitate identification during the surgical procedure and helps the surgeon distinguish between the functional components of the construct.

The system can be provided in several size configurations to accommodate different anatomical requirements and bone quality conditions. A first size configuration is the 1.45 mm rigid anchor, which utilizes a coreless ultra-high molecular weight polyethylene (UHMWPE) #2 MaxBraid™ suture zip strand. The 1.45 mm anchor is mounted on a short inserter that does not require a drill guide, making it particularly suited for open small bone and joint procedures where direct visualization is available. This version utilizes a 1.45 mm drill bit to create the pilot hole. A second size configuration is the 1.5 mm anchor, which also uses a coreless UHMWPE #2 MaxBraid™ suture zip strand. The 1.5 mm anchor shares instrumentation with the 1.5 mm all-suture anchor portfolio, which simplifies inventory management and allows surgeons familiar with the system to utilize the same instruments. A third size configuration includes the 2.9 mm and 2.9 mm self-punching (SP) versions, which utilize a coreless UHMWPE #5 MaxBraid™ suture zip strand. These larger anchors are available on both arthroscopic and self-punching inserters, with the self-punching version eliminating the need for a separate drilling step in bone of appropriate quality. The 2.9 mm anchors are compatible with instrumentation used for 2.9 mm anchors. The 2.9 mm anchor is capable of accepting up to three limbs of #2 MaxBraid™ sutures or 1.5 mm BroadBand tapes, providing flexibility in the repair construct design.

The instrumentation for the implantation procedure can include both disposable sterile components and reusable non-sterile components. For the 1.5 mm anchor system, disposable instrumentation includes a straight kit containing a guide, obturator, and drill bit. A curved kit can also be available, containing a curved guide, obturator, and drill bit. A curved kit with centering sleeve provides additional guidance during the drilling process. A centering sleeve kit is available separately and includes a centering sleeve guide, a centering sleeve insert, and the centering sleeve itself. Additional disposable components include a large shank drill bit, a flexible drill bit, a flexible trocar sized for 1.4 mm and 1.5 mm applications, and a flexible obturator also sized for 1.4 mm and 1.5 mm applications. Reusable instrumentation for the 1.5 mm system can include a straight guide, a curved guide, a straight trocar, a percutaneous trocar for minimally invasive approaches, and an obturator. These reusable instruments are non-sterile and must be sterilized prior to use according to established protocols.

For the 2.9 mm anchor system, disposable instrumentation can include a drill bit, a straight kit containing a guide, obturator, and drill bit, and a short drill bit with needles. Reusable instrumentation for the 2.9 mm system optionally includes a drill guide, an obturator, a trocar, and an in-guide punch. The in-guide punch may be used to create the pilot hole in bone of appropriate quality without the need for a powered drill. The sharing of instrumentation between the anchor systems described herein at both the 1.5 mm and 2.9 mm sizes represents a design consideration that reduces the number of unique instruments required in the operating room and simplifies the learning curve for surgeons transitioning between anchor types.

The surgical technique for implanting the JuggerLoop all-suture anchor and completing a rotator cuff repair begins with preparation of the bone surface at the intended fixation site. The surgeon may roughen the bone surface as desired to create a bleeding bone bed, which may promote biological healing at the anchor site. However, it is important that the cortical bone integrity is not compromised at the anchor sites, as the cortical bone provides the primary resistance to anchor pullout. If the cortical bone is damaged or weakened during surface preparation, the anchor may not achieve adequate fixation strength.

Following bone surface preparation, the drill guide is placed securely and perpendicular to the bone at the desired surgeon-selected location. The perpendicular orientation of the guide ensures that the pilot hole will be created at an appropriate angle to the bone surface, which affects the deployment of the anchor and the direction of suture tension during the repair. The guide should be held firmly against the bone throughout the drilling process to prevent movement that could result in an oversized or misdirected pilot hole.

A drill bit is then inserted into a power drill. The drill bit is advanced into the drill chuck up to a proximal laser-etch line marked on the drill bit shaft. This laser-etch line serves as a depth indicator, ensuring that the drill bit will create a pilot hole of the appropriate depth when fully advanced. Creating a pilot hole of the correct depth is important because a hole that is too shallow may not allow full deployment of the anchor, while a hole that is too deep may weaken the bone or allow the anchor to be over-inserted. With the drill bit properly positioned in the drill chuck, the drill is inserted into the drill guide. The surgeon then advances the drill under power until the drill bit makes contact with the guide, indicating that the pilot hole has been created to the specified depth.

Following the creation of the pilot hole, the drill is removed from the guide while the guide is held firmly in position over the pilot hole. Maintaining precise guide position is advantageous because the anchor must enter the pilot hole along the same trajectory that was used during drilling. If the guide shifts position between drilling and anchor insertion, the anchor may not seat properly within the pilot hole. With the guide held firmly against the bone, the all-suture anchor such as those described herein is inserted through the guide and into the pilot hole. The anchor is mounted on an inserter, and the inserter handle is lightly malleted to advance the anchor into the bone. The malleting continues until a laser etch line on the inserter is flush with the back of the inserter handle. This laser etch line serves as a visual indicator that the anchor has been advanced to the proper depth within the pilot hole. Caution must be taken not to mallet the implant further than the etch line, as over-insertion could cause the guide to contact and weaken the surrounding bone, potentially compromising the fixation.

Once the anchor has been fully seated at the proper depth, the anchor deployment sequence begins. The inserter handle is lightly pulled back to begin setting the anchor within the bone. This initial pull initiates the expansion or reconfiguration of the anchor structure from its insertion configuration to its deployed configuration. The deployed configuration provides resistance to pullout by engaging the walls of the pilot hole or the surrounding bone tissue. After initiating deployment, the sutures are released from the inserter handle by removing a white suture cap that secures the sutures during the insertion process. With the sutures released, the anchor inserter handle is pulled directly out from the guide. As the inserter is withdrawn, slight upward tension is applied to the sutures to continue setting the anchor within the bone. The drill guide is then removed from the surgical site, leaving the anchor deployed within the pilot hole with the suture elements extending from the bone surface.

Final setting of the anchor is achieved by applying tension to all sutures together. The surgeon grasps all of the suture limbs extending from the anchor and applies a slow and steady pull. This pulling action completes the deployment of the anchor and ensures that the anchor structure is fully engaged with the surrounding bone. It is recommended to pull slowly and steadily to properly deploy the anchor, as a quick or jerking pull could lead to improper setting of the anchor or premature failure of the deployment mechanism. After the anchor has been fully set, verification of the deployment is performed by confirming that the white and blue shuttle strand slides freely through the construct. This sliding capability is necessary for the subsequent loading of repair sutures or tapes. Only the white and blue shuttle strand should be verified for sliding, as the other suture components serve different functions in the construct and may not be designed to slide after deployment.

If the surgical plan calls for multiple medial row anchors, the drilling and insertion steps described above are repeated for each additional anchor. In a typical double-row rotator cuff repair, two medial row anchors may be placed to provide multiple fixation points along the medial aspect of the rotator cuff footprint. The spacing and positioning of multiple anchors depends on the size and configuration of the rotator cuff tear and the surgeon's preferred repair strategy.

With the medial row anchor or anchors deployed and verified, additional repair materials are passed through the anchor construct. The repair materials may include MaxBraid sutures, BroadBand tapes, ActivBraid collagen co-braids, or other compatible suture or tape materials. These additional strands are referred to as added repair strands because they are added to the anchor construct after the anchor has been deployed. The added repair strands are folded into the white shuttle loop that extends from the anchor. Approximately five to six inches of each free suture is positioned within the shuttle loop to ensure adequate length for secure shuttling without premature release. If the repair strands include attached needles, the needles must be cut off prior to shuttling to prevent interference with the shuttling mechanism or damage to the suture materials.

With the added repair strands positioned within the shuttle loop, tension is applied to the white and blue tape side of the shuttle strand. This tension pulls the shuttle loop, along with the added repair strands folded within it, through the anchor body and into the adjustable loop located at the anchor. The shuttling action delivers the added repair strands to a position where they can be captured and secured by the cinching mechanism. This process is repeated for each anchor if multiple medial row anchors have been placed.

The fixation of the added repair strands is secured by tensioning the blue and black cinching strand, also referred to as the zip strand. The surgeon grasps the blue and black suture limb and applies tension by pulling. This pulling action reduces the adjustable loop, drawing it tighter around the added repair strands and burying the free sutures into the bone. As the cinching strand is being tensioned and the adjustable loop is being reduced, the loaded sutures continue to slide through the loop. This sliding capability allows the surgeon to adjust the position and tension of the repair strands as needed before the cinching is complete. It is recommended to pull the zip strand slowly to properly reduce the adjustable loop without damaging the suture materials or prematurely locking the mechanism.

Once the zip strand is fully tensioned and the adjustable loop is fully reduced, the cinching strand becomes static and no longer slides. This transition from a sliding to a static configuration indicates that the cinching is complete and the added repair strands are securely captured within the anchor construct. The remaining blue and black cinching strand that extends from the anchor can be incorporated into the soft tissue repair if additional fixation points are desired, or it can be cut and discarded if it is not needed for the repair construct.

With the medial row fixation complete and the repair strands secured within the anchor constructs, the sutures extending from the anchors are passed through the soft tissue of the rotator cuff. The surgeon uses a suture passer of the preferred type to penetrate the rotator cuff tissue and retrieve the suture limbs. Various suture passing devices and techniques may be employed, including arthroscopic suture passers, penetrating graspers, or shuttle relay techniques. The specific suture passing pattern depends on the configuration of the rotator cuff tear and the surgeon's preferred repair strategy.

In a double-row rotator cuff repair configuration, the sutures passed through the tissue are used to reduce the rotator cuff to the medial row fixation points. Arthroscopic knots may be tied to repair the tendon, with the knot tying proceeding from a posterior to anterior direction across the repair site. However, in a double-row repair, the suture strands are not cut after the knots are tied. Instead, these strands are left intact and are spanned over the lateral aspect of the tendon. The spanning sutures will be secured to the lateral row using knotless anchors, creating a suture bridge configuration that distributes load across the repaired tendon.

The lateral row fixation is achieved using knotless anchors such as the Quattro Link™ or Ventix Link™ anchors. These knotless anchors are designed to capture and secure multiple suture limbs without the need for arthroscopic knot tying. The Ventix Link™ and Quattro Link™ knotless anchors can accommodate up to six limbs of Size 1 or Size 2 MaxBraid or BroadBand suture. The suture limbs spanning from the medial row are loaded into the knotless anchor according to the surgical technique for that specific anchor type. The knotless anchor is then implanted into the lateral aspect of the greater tuberosity, securing the spanning sutures and completing the suture bridge construct. The procedure is finalized by cutting any remaining or excess suture material that extends from the repair site.

The system can be compatible with various suture and tape constructs that may be used as the added repair strands. ActivBraid collagen co-braid options include collagen-UHMWPE co-braid sutures in USP size 2, which are available with half-circle taper point needles in white and black or blue color configurations. ActivBraid collagen-UHMWPE co-braid tapes are available in 1.5 mm width with half-circle taper point needles in white and black or blue color configurations. ActivBraid collagen-UHMWPE co-braid tapes are also available in 2.5 mm width in white and black or blue color configurations without needles. The ActivBraid products are packaged according to specified box quantities but are billed individually.

BroadBand suture tape options include 1.5 mm width tapes, which correspond to a #2 suture size designation. These tapes are available in black and blue or black color configurations, with or without attached needles. Needle options for the 1.5 mm tapes include MO-4 and MO-6 needle configurations. A combination pack containing one black and blue tape and one black tape is also available. BroadBand suture tape options also include 2.3 mm width tapes, which correspond to a #5 suture size designation. These tapes are available in black and blue, black, or black and white color configurations, with or without attached needles. Needle options for the 2.3 mm tapes include CTX and CCS needle configurations. A combination pack containing one black and blue tape with cutting needles and one black tape with cutting needles is also available.

MaxBraid suture options include USP size 2 sutures, which are available in black and blue, blue, or black color configurations. These sutures are available with or without attached needles, with needle options including MO-4 and MO-6 configurations. Combination packs containing black and blue and blue sutures are available with or without needles. MaxBraid suture options also include USP size 5 sutures, which are available in black and blue and blue color configurations with MO-6 or CCS needles.

The implant catalog for the OsseoCoat JuggerLoop anchors includes three configurations. A first configuration is the OsseoCoat JuggerLoop 1.5 mm with MaxBraid, which provides the osteoconductive coating on a 1.5 mm diameter anchor. A second configuration is the OsseoCoat JuggerLoop 2.9 mm with MaxBraid, which provides the osteoconductive coating on a 2.9 mm diameter anchor designed for use with a drilled pilot hole. A third configuration is the OsseoCoat JuggerLoop 2.9 mm with MaxBraid in a self-punching version, which provides the osteoconductive coating on a 2.9 mm diameter anchor designed to create its own pilot hole during insertion without the need for a separate drilling step.

The various components of the system are manufactured by multiple entities. Biomet Sports Medicine, located in Warsaw, Indiana, is the legal manufacturer of the system and associated disposable and reusable instrumentation that is shared with the anchor system. Cayenne Medical Inc., located in Scottsdale, Arizona, is the legal manufacturer of the Ventix Link™ knotless anchors and Quattro Link knotless anchors that may be used for lateral row fixation in double-row repairs. Embody, Inc., located in Norfolk, Virginia, is the legal manufacturer of the ActivBraid™ collagen co-braid products that are compatible with the anchor system. Riverpoint Medical, located in Portland, Oregon, is the legal manufacturer of the JuggerLoop soft anchors themselves, as well as the MaxBraid suture and BroadBand tape products.

The scientific foundation for the osteoconductive properties of the OsseoCoat technology is supported by published literature. Histomorphometric studies have compared hydroxyapatite coated and uncoated porous titanium bone implants, demonstrating differences in bone integration between coated and uncoated surfaces. Studies of surface characteristics and biological responses have examined hydroxyapatite coatings applied by various methods, providing data on the cellular and tissue responses to these coatings. Comprehensive reviews of bioactive glass materials have traced the development of these materials from early formulations to modern hybrid compositions, documenting their interactions with bone tissue and their potential to promote bone formation. These scientific references provide support for the use of hydroxyapatite and bioceramic coatings on orthopedic implants intended for bone fixation applications.

The JuggerLoop all-suture anchor system thus provides a comprehensive solution for soft tissue to bone fixation in rotator cuff repair and other orthopedic procedures. The anchor construct incorporates a shuttle mechanism for passing additional repair materials, a cinching mechanism for securing those materials within the bone, and an optional osteoconductive coating for promoting bone integration. The availability of multiple anchor sizes accommodates different anatomical requirements, while the sharing of instrumentation with the JuggerKnot anchor portfolio simplifies surgical workflow. The compatibility with various suture and tape constructs, including collagen co-braids, provides flexibility in repair construct design. The surgical technique described herein provides a systematic approach to anchor placement, suture loading, and repair completion that can be adapted to various tear configurations and surgical preferences.

According to one example, amethod of repairing soft tissue to bone, the method comprising: placing a drill guide perpendicular to a bone surface at a desired fixation location; drilling a pilot hole through the drill guide to a predetermined depth using a drill bit having a laser-etch line indicating the predetermined depth; inserting an all-suture anchor through the drill guide and into the pilot hole, wherein the all-suture anchor comprises a shuttle pull strand, a shuttle loop, a cinching strand, and an adjustable loop; deploying the all-suture anchor within the pilot hole by pulling back on an inserter handle; releasing sutures from the inserter handle by removing a suture cap; removing the inserter handle and the drill guide from the bone surface; setting the all-suture anchor by applying tension to all sutures extending from the anchor; verifying that the shuttle pull strand slides through the anchor construct; passing at least one repair strand through the shuttle loop; shuttling the at least one repair strand into the adjustable loop by applying tension to the shuttle pull strand; cinching the adjustable loop by applying tension to the cinching strand to secure the at least one repair strand within the bone; passing the at least one repair strand through soft tissue; and securing the soft tissue to the bone using the at least one repair strand.

Example 2. The method of Example 1, further comprising roughening the bone surface prior to placing the drill guide to create a bleeding bone surface while maintaining cortical bone integrity at the fixation location.

Example 3. The method of Example 1, wherein inserting the all-suture anchor comprises malleting the inserter handle until a laser etch line on the inserter is flush with a back of the inserter handle.

Example 4. The method of Example 1, wherein setting the all-suture anchor comprises applying a slow and steady pull on all sutures together.

Example 5. The method of Example 1, wherein the at least one repair strand comprises at least one of a MaxBraid suture, a BroadBand tape, or an ActivBraid collagen co-braid.

Example 6. The method of Example 1, wherein passing the at least one repair strand through the shuttle loop comprises folding approximately five to six inches of the at least one repair strand into the shuttle loop.

Example 7. The method of Example 1, wherein the at least one repair strand comprises an attached needle, and wherein the method further comprises cutting the attached needle from the at least one repair strand prior to passing the at least one repair strand through the shuttle loop.

Example 8. The method of Example 1, wherein cinching the adjustable loop comprises applying a slow pull on the cinching strand to reduce the adjustable loop.

Example 9. The method of Example 1, wherein the cinching strand becomes static after the adjustable loop is fully cinched.

Example 10. The method of Example 9, further comprising incorporating the static cinching strand into a soft tissue repair or cutting and discarding the static cinching strand.

Example 11. The method of Example 1, wherein the all-suture anchor comprises a coreless ultra-high molecular weight polyethylene suture zip strand.

Example 12. The method of Example 1, wherein the all-suture anchor comprises an OsseoCoat technology coating having hydroxyapatite and bioceramic particles embedded in an anchor portion of the all-suture anchor.

Example 13. The method of Example 1, wherein the all-suture anchor has a diameter of 1.45 mm, 1.5 mm, or 2.9 mm.

Example 14. The method of Example 1, wherein the all-suture anchor has a diameter of 2.9 mm and wherein the method comprises passing up to three limbs of repair strands through the shuttle loop.

Example 15. The method of Example 1, wherein securing the soft tissue to the bone comprises tying arthroscopic knots to secure the at least one repair strand to the soft tissue.

Example 16 is a method of performing a double-row rotator cuff repair, the method comprising: placing a first drill guide perpendicular to a bone surface at a first medial row fixation location; drilling a first pilot hole through the first drill guide; inserting a first all-suture anchor into the first pilot hole, wherein the first all-suture anchor comprises a first shuttle pull strand, a first shuttle loop, a first cinching strand, and a first adjustable loop; deploying the first all-suture anchor within the first pilot hole; setting the first all-suture anchor by applying tension to sutures extending from the first all-suture anchor; placing a second drill guide perpendicular to the bone surface at a second medial row fixation location; drilling a second pilot hole through the second drill guide; inserting a second all-suture anchor into the second pilot hole, wherein the second all-suture anchor comprises a second shuttle pull strand, a second shuttle loop, a second cinching strand, and a second adjustable loop; deploying the second all-suture anchor within the second pilot hole; setting the second all-suture anchor by applying tension to sutures extending from the second all-suture anchor; passing at least one first repair strand through the first shuttle loop and shuttling the at least one first repair strand into the first adjustable loop; cinching the first adjustable loop by applying tension to the first cinching strand; passing at least one second repair strand through the second shuttle loop and shuttling the at least one second repair strand into the second adjustable loop; cinching the second adjustable loop by applying tension to the second cinching strand; passing the at least one first repair strand and the at least one second repair strand through rotator cuff tissue; tying arthroscopic knots from posterior to anterior to secure the rotator cuff tissue to the medial row without cutting suture strands; spanning the suture strands over a lateral aspect of the rotator cuff tissue; and securing the suture strands at a lateral row using at least one knotless anchor.

Example 17. The method of Example 16, wherein the at least one knotless anchor comprises a Quattro Link knotless anchor or a Ventix Link knotless anchor.

Example 18. The method of Example 16, wherein securing the suture strands at the lateral row comprises loading up to six suture limbs into the at least one knotless anchor.

Example 19. The method of Example 16, further comprising cutting remaining or excess suture after securing the suture strands at the lateral row.

Example 20. The method of Example 16, wherein the first all-suture anchor and the second all-suture anchor each comprise an OsseoCoat technology coating having hydroxyapatite and bioceramic particles providing osteoconductive properties.

Example 21 is a method of loading repair strands into a deployed all-suture anchor, the method comprising: providing an all-suture anchor deployed within a bone pilot hole, the all-suture anchor comprising a shuttle pull strand having a first color combination, a shuttle loop, a cinching strand having a second color combination different from the first color combination, and an adjustable loop; verifying that the shuttle pull strand slides through the all-suture anchor; folding at least one repair strand into the shuttle loop, wherein the at least one repair strand has a length of approximately five to six inches folded into the shuttle loop; applying tension to the shuttle pull strand to pass the at least one repair strand through the all-suture anchor and into the adjustable loop; applying tension to the cinching strand to reduce the adjustable loop and secure the at least one repair strand within the bone pilot hole; and confirming that the at least one repair strand slides through the reduced adjustable loop while the cinching strand is being tensioned.

Example 22. The method of Example 21, wherein the first color combination comprises white and blue and the second color combination comprises blue and black.

Example 23.The method of Example 21, wherein the shuttle loop is white.

Example 24. The method of Example 21, wherein the at least one repair strand comprises up to three limbs of suture or tape when the all-suture anchor has a diameter of 2.9 mm.

Example 25. The method of Example 21, wherein the cinching strand becomes static and non-sliding after the adjustable loop is fully reduced.

Example 26. The method of Example 21, further comprising removing attached needles from the at least one repair strand prior to folding the at least one repair strand into the shuttle loop.

Example 27. The method of Example 21, wherein the at least one repair strand comprises a collagen-UHMWPE co-braid construct.

Example 28 is a method of implanting an all-suture anchor, the method comprising: positioning a drill guide against a bone surface in a perpendicular orientation; inserting a drill bit into a power drill to a proximal laser-etch line on the drill bit; advancing the drill bit through the drill guide until the drill bit contacts the drill guide to create a pilot hole of a predetermined depth; removing the drill bit while maintaining the drill guide in position over the pilot hole; inserting an all-suture anchor on an inserter through the drill guide and into the pilot hole; malleting the inserter until a laser etch line on the inserter is flush with a back of an inserter handle; pulling back on the inserter handle to begin deploying the all-suture anchor; removing a suture cap from the inserter handle to release sutures; withdrawing the inserter handle from the drill guide while applying upward tension on the sutures; removing the drill guide from the bone surface; and applying a slow and steady pull on all sutures together to fully set the all-suture anchor within the pilot hole.

Example 29. The method of Example 28, further comprising verifying deployment of the all-suture anchor by confirming that a shuttle strand slides through the all-suture anchor.

Example 30. The method of Example 28, wherein the all-suture anchor comprises a self-punching configuration that creates the pilot hole during insertion without a separate drilling step.

Example 31. The method of Example 28, wherein the drill guide comprises a straight guide or a curved guide.

Example 32. The method of Example 28, wherein the drill guide comprises a centering sleeve, a centering sleeve guide, and a centering sleeve insert.

Example 33. The method of Example 28, wherein malleting the inserter further than the laser etch line is avoided to prevent the drill guide from weakening the bone.

Example 34 is a method of completing a soft tissue repair using an all-suture anchor system, the method comprising: deploying an all-suture anchor within a bone pilot hole at a medial row location, the all-suture anchor having a shuttle mechanism and a cinching mechanism; loading at least one repair strand into the all-suture anchor using the shuttle mechanism; securing the at least one repair strand within the bone using the cinching mechanism; passing the at least one repair strand through soft tissue using a suture passer; tying the at least one repair strand to the soft tissue; spanning untrimmed portions of the at least one repair strand over a lateral aspect of the soft tissue; and implanting a knotless anchor at a lateral row location to secure the untrimmed portions of the at least one repair strand.

Example 35. The method of Example 34, wherein the knotless anchor receives up to six suture limbs.

Example 36. The method of Example 34, wherein tying the at least one repair strand comprises tying arthroscopic knots from posterior to anterior.

Example 37. The method of Example 34, further comprising trimming excess suture after implanting the knotless anchor.

Example 38. The method of Example 34, wherein the soft tissue comprises rotator cuff tissue.

Example 39. The method of Example 34, wherein the shuttle mechanism comprises a shuttle pull strand and a shuttle loop, and wherein the cinching mechanism comprises a cinching strand and an adjustable loop.

Example 40. The method of Example 34, wherein loading the at least one repair strand comprises folding the at least one repair strand into the shuttle loop and pulling on the shuttle pull strand to pass the at least one repair strand into an adjustable loop at the all-suture anchor.

Many of the FIGURES herein illustrate components of the tissue repair devices including the anchors in a highly schematic manner. This is done to better illustrate interaction of various suture strand constructs. However, it is recognized that the deformable anchors can have other shapes and can be deformable as discussed above. Similarly, other components can have shapes different from those illustrated herein.

It will be understood that the foregoing are merely examples, and that other deployment schemes and device configurations can also be used. Furthermore, combinations of the various foregoing examples can be used together as desired according to further contemplated examples. It will be understood that one of ordinary skill in the art can modify the foregoing devices to achieve a desirable deployment of the first anchor and the second anchor.

While this invention has been described as having example designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### Claims Related Examples

To further illustrate the devices and related methods disclosed herein, a non-limiting list of examples (referred to as aspects and techniques) is provided below. Each of the following non-limiting examples can stand on its own, or can be combined in any permutation or combination with any one or more of the other examples.

In some aspects, the techniques described herein relate to an assembly for securing soft tissue to bone, optionally including: a flexible tubular anchor having a passage therethrough, wherein the flexible tubular anchor is configured for insertion into the bone; a first suture strand having an eyelet, wherein the first suture strand is configured to pass through at least a portion of the passage; and a second suture strand coupled to the flexible tubular anchor and configured to be received by the eyelet, wherein the second suture strand passes through at least a portion of the passage and includes a loop, and wherein the first suture strand is configured to shuttle a free portion of the second suture strand or a third suture strand through the loop, wherein the loop is adjustable to change a size thereof and is configured to capture the free portion of the second suture strand or the third suture strand to couple the free portion of the second suture strand or the third suture strand to the flexible tubular anchor.

In some aspects, the techniques described herein relate to an assembly, wherein the loop can be captured by the flexible tubular anchor within the passage, wherein the first suture strand can be configured to shuttle the free portion of the second suture strand through the loop, wherein the loop can be adjustable to close about itself and thereby cinch and capture the free portion of the second suture strand within the flexible tubular anchor.

In some aspects, the techniques described herein relate to an assembly, wherein the loop extends from the flexible tubular anchor, and wherein the second suture strand optionally includes a second portion that acts to change the size of the loop.

In some aspects, the techniques described herein relate to an assembly, wherein the loop can be adjustable to close about itself and thereby cinch and capture the third suture strand with the flexible tubular anchor.

In some aspects, the techniques described herein relate to an assembly, wherein at least one of the second suture strand or the third suture strand optionally includes a bulked section and a non-bulked section, wherein the bulked section includes a relatively larger cross-sectional area than the non-bulked section.

In some aspects, the techniques described herein relate to an assembly, wherein the bulked section optionally includes a biological material in combination with a synthetic material.

In some aspects, the techniques described herein relate to an assembly, wherein the biological material can be captured within or forms an outer braid of the bulked section.

In some aspects, the techniques described herein relate to an assembly, wherein the biological material can be exposed through openings in or is entirely encapsulated by the synthetic material.

In some aspects, the techniques described herein relate to an assembly, wherein the second suture strand can be a repair strand that is coupled to the soft tissue.

In some aspects, the techniques described herein relate to a method of securing tissue to bone, optionally including: providing a flexible tubular anchor having cinching strand coupled thereto, wherein the cinching strand forms a loop extending from and positioned substantially entirely externally to the flexible tubular anchor; passing a repair suture strand through the loop with a shuttle strand; adjusting a size of the loop using a free portion of the cinching strand; and inserting the flexible tubular anchor into the bone.

In some aspects, the techniques described herein relate to a method, further optionally including securing the repair suture strand to a second anchor and inserting the second anchor into the bone.

In some aspects, the techniques described herein relate to a method, wherein the passing the repair suture strand through the loop with the shuttle strand and the adjusting the size of the loop can be performed before, during, or after the inserting the flexible tubular anchor into the bone.

In some aspects, the techniques described herein relate to a method, further optionally including: providing a second flexible tubular anchor having the repair suture strand coupled thereto; inserting the second flexible tubular anchor into the bone; passing the repair suture strand through the loop; and securing the repair suture strand to the second flexible tubular anchor.

In some aspects, the techniques described herein relate to a method, wherein the securing the repair suture strand to the second flexible tubular anchor optionally includes closing a second loop about itself to cinch and capture the repair suture strand within the second flexible tubular anchor.

In some aspects, the techniques described herein relate to a method, wherein the securing the repair suture strand to the second flexible tubular anchor optionally includes: providing a second cinching strand coupled to the second flexible tubular anchor, wherein the second cinching strand forms a second loop extending from the second flexible tubular anchor; passing the repair suture strand through the second loop with a shuttle strand; adjusting the size of the second loop using a free portion of the second cinching strand; and inserting the second flexible tubular anchor into the bone.

In some aspects, the techniques described herein relate to a method, wherein at least one of the repair suture strand optionally includes a bulked section and a non-bulked section, wherein the bulked section includes a relatively larger cross-sectional area than the non-bulked section, and wherein the bulked section optionally includes a biological material in combination with a synthetic material.

In some aspects, the techniques described herein relate to a method, further optionally including cinching and capturing the repair suture strand with a third loop captured by one of the flexible tubular anchor or the second flexible tubular anchor.

In some aspects, the techniques described herein relate to a method of performing a rotator cuff repair optionally including: inserting a first flexible tubular anchor and second flexible tubular anchor in a medial row; inserting a third flexible tubular anchor and a fourth flexible tubular anchor in a lateral row; passing a first repair strand from the third flexible tubular anchor through an adjustable loop coupled to and extending from the first flexible tubular anchor or passing a second repair strand from the first flexible tubular anchor through an adjustable loop coupled to and extending from the third flexible tubular anchor; and securing the first repair strand to the fourth flexible tubular anchor or securing the second repair strand to the second flexible tubular anchor.

In some aspects, the techniques described herein relate to a method, optionally further including: passing a third flexible tubular anchor from the fourth flexible tubular anchor through an adjustable loop coupled to and extending from the second flexible tubular anchor or passing a fourth repair strand from the second flexible tubular anchor through an adjustable loop coupled to and extending from fourth flexible tubular anchor; and securing the third repair strand to the third flexible tubular anchor or securing the fourth repair strand to the first flexible tubular anchor.

In some aspects, the techniques described herein relate to a method, optionally further including adjusting a size of the loop coupled to and extending from the first flexible tubular anchor and the loop coupled to and extending from the third flexible tubular anchor before, during, or after the inserting the first flexible tubular anchor into the medial row and the third flexible tubular anchor into the lateral row.

In some aspects, the techniques described herein relate to a method of performing a rotator cuff repair optionally including: inserting at least a first flexible tubular anchor in a medial row; inserting at least a second flexible tubular anchor in a lateral row; passing a first repair strand from the second flexible tubular anchor through an adjustable loop coupled to and extending from the first flexible tubular anchor; and securing the first repair strand to the second flexible tubular anchor.

What is claimed is:

1. An assembly for securing soft tissue to bone, comprising:
a flexible tubular anchor having a passage therethrough, wherein the flexible tubular anchor is configured for insertion into the bone;
a first suture strand having an eyelet, wherein the first suture strand is configured to pass through at least a portion of the passage; and
a second suture strand coupled to the flexible tubular anchor and configured to be received by the eyelet, wherein the second suture strand passes through at least a portion of the passage and includes a loop, and wherein the first suture strand is configured to shuttle a free portion of the second suture strand or a third suture strand through the loop, wherein the loop is adjustable to change a size thereof and is configured to capture the free portion of the second suture strand or the third suture strand to couple the free portion of the second suture strand or the third suture strand to the flexible tubular anchor.

2. The assembly of claim 1, wherein the loop is captured by the flexible tubular anchor within the passage, wherein the first suture strand is configured to shuttle the free portion of the second suture strand through the loop, wherein the loop is adjustable to close about itself and thereby cinch and capture the free portion of the second suture strand within the flexible tubular anchor.

3. The assembly of claim 1, wherein the loop extends from the flexible tubular anchor, and wherein the second suture strand includes a second portion that acts to change the size of the loop.

4. The assembly of claim 3, wherein the loop is adjustable to close about itself and thereby cinch and capture the third suture strand with the flexible tubular anchor.

5. The assembly of claim 1, wherein at least one of the second suture strand or the third suture strand includes a bulked section and a non-bulked section, wherein the bulked section includes a relatively larger cross-sectional area than the non-bulked section.

6. The assembly of claim 5, wherein the bulked section includes a biological material in combination with a synthetic material.

7. The assembly of claim 6, wherein the biological material is captured within or forms an outer braid of the bulked section.

8. The assembly of claim 6, wherein the biological material is exposed through openings in or is entirely encapsulated by the synthetic material.

9. The assembly of claim 1, wherein the second suture strand is a repair strand that is coupled to the soft tissue.

10. A method of securing tissue to bone, comprising:
providing a flexible tubular anchor having cinching strand coupled thereto, wherein the cinching strand forms a loop extending from and positioned substantially entirely externally to the flexible tubular anchor;
passing a repair suture strand through the loop with a shuttle strand;
adjusting a size of the loop using a free portion of the cinching strand; and
inserting the flexible tubular anchor into the bone.

11. The method of claim 10, further comprising securing the repair suture strand to a second anchor and inserting the second anchor into the bone.

12. The method of claim 10, wherein the passing the repair suture strand through the loop with the shuttle strand and the adjusting the size of the loop is performed before, during, or after the inserting the flexible tubular anchor into the bone.

13. The method of claim 10, further comprising:
providing a second flexible tubular anchor having the repair suture strand coupled thereto;
inserting the second flexible tubular anchor into the bone;
passing the repair suture strand through the loop; and
securing the repair suture strand to the second flexible tubular anchor.

14. The method of claim 13, wherein the securing the repair suture strand to the second flexible tubular anchor includes closing a second loop about itself to cinch and capture the repair suture strand within the second flexible tubular anchor.

15. The method of claim 13, wherein the securing the repair suture strand to the second flexible tubular anchor includes:
providing a second cinching strand coupled to the second flexible tubular anchor, wherein the second cinching strand forms a second loop extending from the second flexible tubular anchor;
passing the repair suture strand through the second loop with a shuttle strand;
adjusting the size of the second loop using a free portion of the second cinching strand; and
inserting the second flexible tubular anchor into the bone.

16. The method of claim 13, wherein at least one of the repair suture strand includes a bulked section and a non-bulked section, wherein the bulked section includes a relatively larger cross-sectional area than the non-bulked section, and wherein the bulked section includes a biological material in combination with a synthetic material.

17. The method of claim 13, further comprising cinching and capturing the repair suture strand with a third loop captured by one of the flexible tubular anchor or the second flexible tubular anchor.

18. A method of performing a rotator cuff repair comprising:
inserting a first flexible tubular anchor and second flexible tubular anchor in a medial row;
inserting a third flexible tubular anchor and a fourth flexible tubular anchor in a lateral row;
passing a first repair strand from the third flexible tubular anchor through an adjustable loop coupled to and extending from the first flexible tubular anchor or passing a second repair strand from the first flexible tubular anchor through an adjustable loop coupled to and extending from the third flexible tubular anchor; and
securing the first repair strand to the fourth flexible tubular anchor or securing the second repair strand to the second flexible tubular anchor.

19. The method of claim 18, further comprising:
passing a third flexible tubular anchor from the fourth flexible tubular anchor through an adjustable loop coupled to and extending from the second flexible tubular anchor or passing a fourth repair strand from the second flexible tubular anchor through an adjustable loop coupled to and extending from fourth flexible tubular anchor; and
securing the third repair strand to the third flexible tubular anchor or securing the fourth repair strand to the first flexible tubular anchor.

20. The method of claim 18, further comprising adjusting a size of the loop coupled to and extending from the first flexible tubular anchor and the loop coupled to and extending from the third flexible tubular anchor before, during, or after the inserting the first flexible tubular anchor into the medial row and the third flexible tubular anchor into the lateral row.
